(19) United States
(12) Patent Application Publication
(10) Pub. No.: US 2010/0317698 A1
(43) Pub. Date: Dec. 16, 2010

(54) Polysubstituted Pyridinylaminoalkylene- and Pyridinyloxyalkylene-Cyclopropanamine Compounds, a Process for Their Preparation and Pharmaceutical Compositions Containing Them

(75) Inventors: Solo Goldstein, Suresnes (FR); Claude Guillonneau, Clamart (FR); Yves Charton, Sceaux (FR); Brian Lockhart, Feucherolles (FR); Pierre Lestage, La Celle St Cloud (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Courbevoie Cedex (FR)

(21) Appl. No.: 12/223,311

(22) PCT Filed: Jan. 30, 2007
(86) PCT No.: PCT/FR2007/000170

§ 371 Date: Jul. 25, 2008

(30) Foreign Application Priority Data
Jan. 30, 2006 (FR) 0600784

(51) Int. Cl. A61K 31/444; C07D 213/65; C07D 401/04; C07D 401/10; C07D 213/72; A61K 31/4439; A61K 31/4418; A61P 25/28; A61P 25/00; A61P 25/04; A61P 25/16; A61P 25/34

(52) U.S. Cl. 514/334; 546/300; 546/257; 546/268.4; 546/304; 546/312; 514/340; 514/351; 514/352

(57) ABSTRACT

Compounds of formula (I):

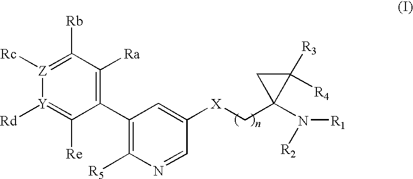

wherein:
n represents an integer of from 1 to 6 inclusive,

X represents an oxygen atom or an NR₆ group,
Y represents a carbon atom or a nitrogen atom,
Z represents a carbon atom or a nitrogen atom,
R₁ and R₂ represent a hydrogen atom or an alkyl or arylalkyl group,
R₃ and R₄ represent a hydrogen atom or an alkyl group,

R₅ represents a hydrogen atom or an alkyl, halogen, hydroxy, alkoxy, cyano, nitro, acyl, alkoxycarbonyl, trihaloalkyl, trihaloalkoxy or optionally substituted amino group,
R₆ represents a hydrogen atom or an alkyl or arylalkyl group,
Ra, Rb, Rc, Rd and Re are as defined in the description.

Medicinal products containing the same which are useful as specific nicotinic ligands of α4β2 receptors.

The present invention relates to new polysubstituted pyridinylaminoalkylene- and pyridinyloxyalkylene-cyclopropanamine compounds, to a process for their preparation and to pharmaceutical compositions containing them.

The compounds of the present invention are especially valuable from a pharmacological point of view because of their specific interaction with central nicotinic receptors of type α4β2, having application in the treatment of neuropathologies associated with cerebral ageing, of mood disorders, of pain and of tobacco withdrawal.

Ageing of the population due to increased life expectancy at birth has brought with it a major increase in the incidence of age-related neuropathologies and especially of Alzheimer's disease. The principal clinical manifestations of cerebral ageing and especially of age-related neuropathologies are deficiencies in mnemic and cognitive functions, which may lead to dementia. It has been widely demonstrated that, of the various neuro-transmitters, acetylcholine plays a major role in memory functions and that there is large-scale destruction of the cholinergic neuronal pathways in certain neurodegenerative diseases or when there is inadequate activation in the case of cerebral ageing. For that reason, numerous therapeutic approaches have been aimed at preventing destruction of the neurotransmitter by means of the inhibition of acetylcholine esterase or have sought to provide a substitute for the deficient neurotransmitter. In the latter case, the cholinergic agonists proposed have been of the muscarinic type, which are specific for post-synaptic M1 receptors.

It has recently been shown that the cholinergic impairment associated with Alzheimer's disease affects neurones carrying nicotinic receptors more than those carrying muscarinic receptors (Schroder et al., “Alzheimer disease: therapeutic strategies”, Birkhauser Boston, 1994, 181-185). Numerous studies have, moreover, demonstrated that nicotine has memory-facilitating properties (Prog. Neuropsychopharmacol., 1992, 16, 181-191) and that these properties are exerted as much on mnemic functions (Psychopharmacol., 1996, 123, 88-97) as on the faculties of attention and vigilance (Psychopharmacol., 1995, 118, 195-205). Furthermore, nicotine exerts neuroprotective effects with respect to excitotoxic agents such as glutamate (Brain Res., 1994, 644, 181-187).

All of these findings can very probably be linked with epidemiological studies that have shown a lower incidence of Alzheimer's disease and Parkinson's disease in smokers. Furthermore, several studies have shown the value of nicotine in the treatment of mood disorders such as states of depression, anxiety or schizophrenia. Finally, it has been shown that nicotine has antalgic properties. All of the therapeutic properties of nicotine and also those described for other nicotinic agents are based upon activity with respect to central receptors, which differ structurally and pharmacologically from peripheral receptors (muscle and ganglion). The central receptors of type α4β2 are the most represented in the central nervous system and have been implicated in the majority of the therapeutic effects of nicotine (Life Sci., 1995, 56, 545-570).

Several documents such as Synlett., 1999, 7, 1053-1054 ; J. Med. Chem, 1985, 28(12), 1953-1957 and 1980, 23(3), 339-341 ; 1970, 13(5), 820-826 ; 1972, 15(10), 1003-1006; J. Am. Chem. Soc., 1987, 109(13), 4036-4046, or a few patents or patent applications such as DE 36 08 727, EP 124 208 or WO 94/10158 describe and claim compounds containing a 1,1- or 1,2-disubstituted cyclopropane moiety. None of those references describe or 1111) 20 suggest that those compounds have pharmacological activity that is specific for nicotinic receptors and, more especially, for central nicotinic receptors of type α4β2, this being a novel property of the compounds described by the Applicant. Patent Application EP 1 170 281 describes 1,1- and 1,2-disubstituted cyclopropane compounds which are nicotinic ligands.

The compounds of the present invention are therefore new and represent powerful selective nicotinic ligands of the central receptor sub-type α4β2. They are consequently of use in the treatment of deficiencies of memory associated with cerebral ageing and with neuro-degenerative diseases such as Alzheimer's disease, Parkinson's disease, Pick's disease, Korsalcoff s disease and frontal lobe and subcortical dementias, and also for the treatment of mood disorders, Tourette's syndrome, attention-deficit hyperactivity syndrome, tobacco withdrawal and pain.

More specifically, the present invention relates to compounds of formula (I):

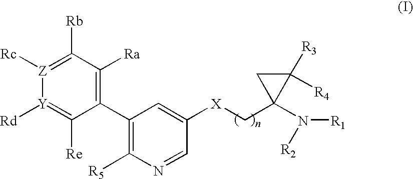

wherein:
n represents an integer of from 1 to 6 inclusive,
X represents an oxygen atom or an NR₆ group,
Y represents a carbon atom or a nitrogen atom, wherein when Y represents a nitrogen atom Rd is absent,
Z represents a carbon atom or a nitrogen atom, wherein when Z represents a nitrogen atom Rc is absent,
R₁ and R₂, which may be identical or different, each independently of the other represent a hydrogen atom, a linear or branched (C₁-C₆)alkyl group or an aryl-(C₁-C₆)alkyl group in which the alkyl moiety may be linear or branched,
R₃ and R₄, which may be identical or different, each independently of the other represent a hydrogen atom or a linear or branched (C₁-C₆)alkyl group,
R₅ represents a hydrogen atom, a linear or branched (C₁-C₆)alkyl, halogen, hydroxy, linear or branched (C₁-C₆)alkoxy, cyano, nitro, linear or branched (C₂-C₆)acyl, linear or branched (C₁-C₆)alkoxycarbonyl, linear or branched (C₁-C₆)trihaloalkyl or linear or branched (C₁-C₆)trihaloalkoxy group, or an amino group optionally substituted by one or two linear or branched (C₁-C₆)alkyl groups, or represents an aryl or heteroaryl group,
R₆ represents a hydrogen atom, a linear or branched (C₁-C₆)alkyl group or an aryl-(C₁-C₆)alkyl group in which the alkyl moiety may be linear or branched,
Ra, Rb, Rc, Rd and Re, which may be identical or different, each independently of the others represent a hydrogen atom, a linear or branched (C₁-C₆)alkyl, halogen, linear or branched (C₁-C₆)haloalkyl, hydroxy, linear or branched (C₁-C₆)alkoxy, linear or branched (C₁-C₆)hydroxyalkyl, cyano, nitro, carboxy, isothiocyanate, linear or branched (C₂-C₆)acyl, linear or branched (C₁-C₆)alkoxycarbonyl, linear or branched (C₁-C₆)trihaloalkyl, linear or branched (C₁-C₆)trihaloalkoxy or linear or branched (C₁-C₆)alkylthio group, a (C₁-C₆)alkylcarbonylamino group in which the alkyl moiety may be linear or branched, a halo-(C₁-C₆)alkylcarbonylamino group in which the alkyl moiety may be linear or branched, an aminocarbonyl group, an amino group optionally substituted by one or two linear or branched (C₁-C₆)alkyl groups, or a tetrazolyl group,

there being understood by aryl group a phenyl, biphenyl, naphthyl, dihydronaphthyl, tetrahydronaphthyl, indanyl or indenyl group, each of those groups being optionally substituted by one or more identical or different groups selected from halogen atoms, linear or branched (C₁-C₆)alkyl, hydroxy, cyano, nitro, linear or branched (C₁-C₆)alkoxy, linear or branched (C₂-C₁)acyl, linear or branched (C₁-C₆)alkoxycarbonyl, linear or branched (C₁-C₆)trihaloalkyl and linear or branched (C₁-C₆)trihaloalkoxy groups and amino groups optionally substituted by one or two linear or branched (C₁-C₆)alkyl groups,

there being understood by heteroaryl group an aromatic monocyclic system or a bicyclic system having from 5 to 12 chain members and containing from one to three identical or different hetero atoms selected from oxygen, nitrogen and sulphur, wherein one of the rings, in the case of the bicyclic system, has an aromatic character while the other ring may be aromatic or partially hydrogenated, and wherein each of those groups may optionally be substituted by one or more identical or different groups selected from the substituents defined above in the case of an aryl group.

According to an advantageous embodiment of the invention, preferred compounds are compounds of formula (I/A):

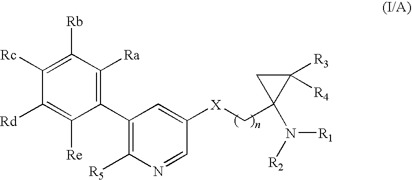

wherein R₁, R₂, R₃, R₄, R₅, Ra, Rb, Rc, Rd, Re, X and n are as defined hereinbefore.

According to a second advantageous embodiment of the invention, preferred compounds are compounds of formula (I/B):

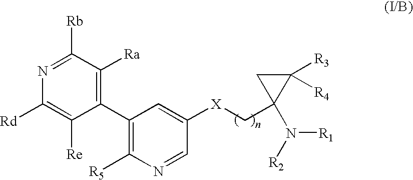

wherein R₁, R₂, R₃, R₄₅ R₅, Ra, Rb, Rd, Re, X and n are as defined hereinbefore.

According to a third advantageous embodiment of the invention, preferred compounds are compounds of formula (I/C):

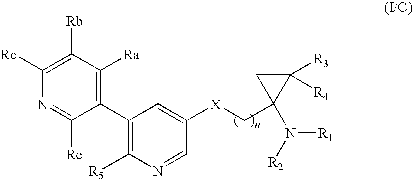

wherein R₁, R₂, R₃, R₄, R₅, Ra, Rb, Rc, Re, X and n are as defined hereinbefore.

Preferred compound of the invention are compounds wherein n is an integer having the value 1.

Preferred substituents R₁ and R₂ according to the invention are the hydrogen atom and the linear or branched (C₁-C₆)alkyl group.

Preferred substituents R₁ and R₂ according to the invention are more especially the hydrogen atom and the methyl group.

Preferred substituents R₃ and R₄ according to the invention are the hydrogen atom and the methyl group.

The preferred substituent R₅ according to the invention is the hydrogen atom, the halogen atom or a linear or branched (C₁-C₆)alkyl group.

The preferred substituent R₆ according to the invention is the hydrogen atom or the methyl group.

Advantageously, preferred compounds of the invention are those wherein Y represents a nitrogen atom and Z represents a carbon atom optionally substituted by Rc.

Advantageously, preferred compounds of the invention are those wherein Y represents a nitrogen atom, Z represents a carbon atom, Ra represents a hydrogen atom, Rb represents a hydrogen atom, Rc represents a hydrogen atom and Re represents a hydrogen atom.

Very advantageously, preferred compounds of the invention are those wherein Y represents a carbon atom optionally substituted by Rd and Z represents a nitrogen atom.

Advantageously, preferred compounds of the invention are those wherein Y represents a carbon atom, Z represents a nitrogen atom, Ra represents a hydrogen atom, Rb represents a hydrogen atom, Rd represents a hydrogen atom and Re represents a hydrogen atom.

The notation (1S,2S), (1R,2R) followed by the name of the compound signifies that the product obtained is a racemic mixture and hence that both configurations are present.

For example:

(1S,2S),(1R,2R)-2-methyl-1-[(3-pyridinyloxy)methyl]cyclopropanamine signifies that the product obtained, a racemic mixture, contains (1S,2S)-2-methyl-1-[(3-pyridinyloxy)-methyl]cyclopropanamine and (1R,2R)-2-ethyl-1-[(3-pyridinyl oxy)methyl]cyclopropanamine.

The notation (R or S) followed by the name of the compound signifies that the product obtained is an optically pure enantiomer. The presence of (−) and/or (+) indicates the sign of the optical rotation.

The notation (R,S) followed by the name of the compound signifies that the product obtained is a racemic mixture and hence that both configurations are present.

The notation (1S,2S) or (1R,2R) followed by the name of the compound signifies that the product obtained is an optically pure enantiomer. The presence of (−) and/or (+) indicates the sign of the optical rotation.

For example:

(1S,25)- or (1R,2R)-(−)-N,2-dimethyl-1-[(3-pyridinyloxy)methyl]cyclopropanamine dihydrochloride signifies that the product obtained, an optically pure enantiomer, is (1S,2S)-(−)-N,2-dimethyl-1-[(3-pyridinyloxy)methyl]cyclopropanamine dihydrochloride or (1R,2R)-(−)-N,2-dimethyl-1-[(3-pyridinyloxy)methyl]cyclopropanamine dihydrochloride

The α and β enantiomers are understood to be the optically pure enantiomers of the racemic mixture in question.

In especially advantageous manner, preferred compounds of the invention are:

[1-({[5-(3-methoxyphenyl)pyridin-3-yl]oxy}methyl)cyclopropyl]methylamine dihydrochloride,

[1-({[6-chloro-5-(3-methoxyphenyl)pyridin-3-yl]oxy}methyl)cyclopropyl]methylamine dihydrochloride,

[1-({[5-(4-methoxyphenyl)pyridin-3-yl]oxy}methyl)cyclopropyl]methylamine dihydrochloride,

[1-({[5-(4-chlorophenyl)pyridin-3-yl]oxy}methyl)cyclopropyl]methylamine hydrochloride,

[1-({[6-chloro-5-(4-fluorophenyl)pyridin-3-yl]oxy}methyl)cyclopropyl]methylamine hydrochloride,

{1-[({6-chloro-5-[4-(methylthio)phenyl]pyridin-3-yl }oxy)methyl]cyclopropyl}methylamine dihydrochloride,

[1-({[6-chloro-5-(3,5-dichlorophenyl)pyridin-3-yl]oxy}methyl)cyclopropyl]methylamine hydrochloride,

N-[3-(2-chloro-5-{[1-(methylamino)cyclopropyl]methoxy}pyridin-3-yl)phenyl]acetamide hydrochloride,

ethyl 4-(2-chloro-5-{[1-(methylamino)cyclopropyl]methoxy}pyridin-3-yl)benzoate dihydrochloride,

4-(2-chloro-5-{[1-(methylamino)cyclopropyl]methoxy}pyridin-3-yl)benzamide hydrochloride,

4-(2-chloro-5-{[1-(methylamino)cyclopropyl]methoxy}pyridin-3-yl)benzoic acid hydrochloride,

(1-{[(2-chloro-3,4′-bipyridin-5-yl)oxy]methyl}cyclopropyl)methylamine dihydrochloride,

{1-[({6-chloro-5-[4-(2H-tetrazol-5-yl)phenyl]pyridin-3-yl}oxy)methyl]cyclopropyl}-methylamine dihydrochloride,

[1-{[5,6-bis(4-chlorophenyl)pyridin-3-yl]oxy}methyl)cyclopropyl]methylamine dihydrochloride,

5-(4-aminophenyl)-6-methyl-N-{[1-(methylamino)cyclopropyl]methyl}pyridin-3-amine trihydrochloride,

The enantiomers and diastereoisomers, as well as the addition salts thereof with a pharmaceutically acceptable acid or base, of the preferred compounds form an integral part of the invention.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

The present invention relates also to a process for the preparation of compounds of formula (I), which is characterised in that there is used as starting material a compound of formula (II):

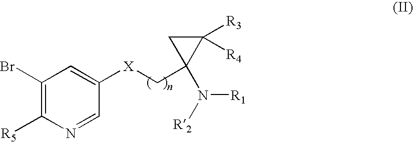

wherein R′₂ represents a hydrogen atom, a methyl group or a tert-butoxycarbonyl group and R₁, R₃, R₄, R₅, X and n are as defined for formula (I), which compounds of formula (II) are reacted with a compound of formula (III):

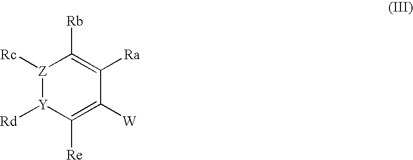

wherein W represents an —Sn(C₄H₉)₃, —B(OH)₂ or

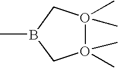

group, and Ra, Rb, Rc, Rd, Re, Y and Z are as defined for formula (I), in the presence of Pd(PPh₃)₄, in basic medium, to yield compounds of formula (IV):

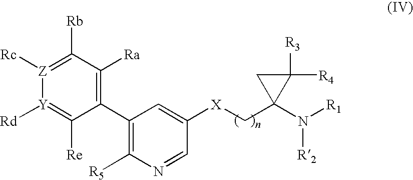

wherein R₁, R′₂, R₃, R₄, R₅, X, Y, Z, Ra, Rb, Rc, Rd, Re and n are as defined hereinbefore,

which compounds of formula (IV), when R′₂ represents a tert-butoxycarbonyl group, are placed in the presence of hydrochloric acid to yield compounds of formula (I/a), a particular case of the compounds of formula (I):

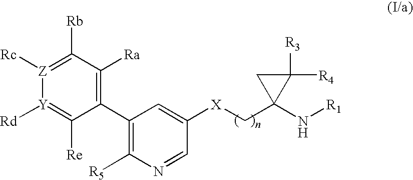

wherein R₁, R₃, R₄, R₅, X, Y, Z, Ra, Rb, Rc, Rd, Re and n are as defined hereinbefore,

which compounds of formula (I/a) are reacted with a compound of formula (V):

R″₂-L₂ (V)

wherein R″₂ represents a linear or branched (C₁-C₆)alkyl group or an aryl-(C₁-C₆)alkyl group in which the alkyl moiety may be linear or branched, and L₂ represents a leaving group customary in organic chemistry, in basic medium, to yield compounds of formula (I/b), a particular case of the compounds of formula (I):

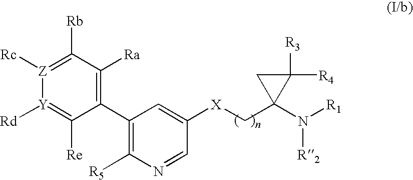

wherein R₁, R″₂, R₃, R₄, R₅, X, Y, Z, Ra, Rb, Rc, Rd, Re and n are as defined hereinbefore,

the totality of the compounds of formulae (I/a) and (I/b) constituting the totality of the compounds of the invention, which are purified, where appropriate, according to conventional purification techniques, which may be separated into their different isomers according to a conventional separation technique, and which are converted, where appropriate, into addition salts thereof with a pharmaceutically acceptable acid or base.

According to one embodiment of the invention, compounds of formula (II), in the case where X represents an oxygen atom, R₃ and R₄ each represent a hydrogen atom and R′₂ represents a tert-butoxycarbonyl group, of formula (II/a):

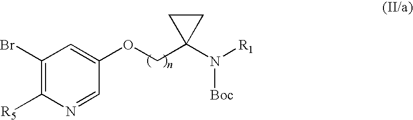

wherein Boc represents a tert-butoxycarbonyl group and R₁, R₅ and n are as defined hereinbefore,

may be prepared starting from a compound of formula (VI):

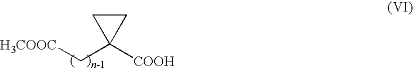

wherein n is as defined hereinbefore, which is reacted with diphenylphosphoryl azide in basic medium and then placed in the presence of tert-butanol to yield compounds of formula (VII):

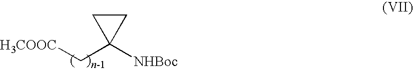

wherein n and Boc are as defined hereinbefore,

which compounds of formula (VII) are reacted with a group of formula (VIII):

R′₁-L₁ (VIII)

wherein R′₁ represents a linear or branched (C₁-C₆)alkyl group or an aryl-(C₁-C₆)alkyl group in which the alkyl moiety may be linear or branched and L₁ represents a leaving group customary in organic chemistry, in basic medium, to yield compounds of formula (IX):

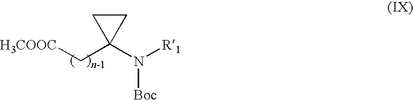

wherein R′₁, Boc and n are as defined hereinbefore, the compounds of formulae (VII) and (IX) constituting the compounds of formula (X):

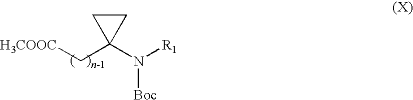

wherein R₁ is as defined for formula (I) and Boc and n are as defined hereinbefore, which compounds of formula (X) are placed in the presence of a reducing agent to yield compounds of formula (XI):

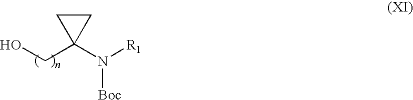

wherein R₁, Boc and n are as defined hereinbefore,

which compounds of formula (XI) are placed in the presence of carbon tetrabromide and triphenylphosphine to yield compounds of formula (XII):

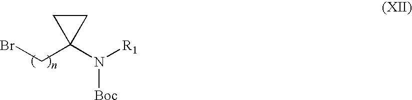

to wherein R₁, Boc and n are as defined hereinbefore, which compounds of formula (XII) are reacted with a compound of formula (XIII):

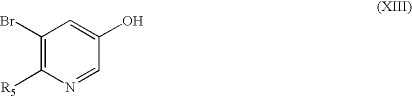

wherein R₅ is as defined for formula (I), in basic medium, to yield compounds of formula (II/a) as defined hereinbefore.

According to another embodiment of the invention, the compounds of formula (II), in the case where X represents an oxygen atom, n has the value 1, R′₂ represents a methyl group and one of the groups R₃ or R₄ represents a methyl group and the other group R₃ or R₄ represents a hydrogen atom, of formula (II/b):

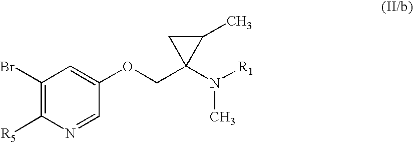

wherein R₁ and R₅ are as defined hereinbefore, may be prepared starting from 1,2-dibromopropane and ethyl isocyanate in basic medium to yield the compound of formula (XIV):

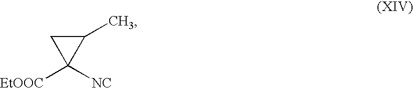

which compound of formula (XIV) is placed in the presence of a reducing agent to yield the compound of formula (XV):

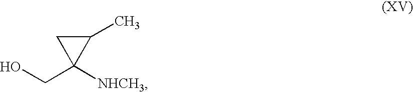

which compound of formula (XV) is reacted with a compound of formula (XVI):

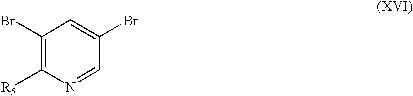

wherein R₅ is as defined hereinbefore, in basic medium, to yield compounds of formula (XVII):

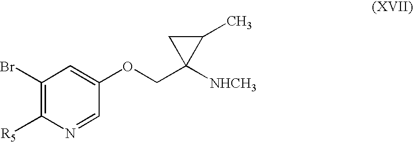

wherein R₅ is as defined hereinbefore,

which compounds of formula (XVII) are reacted with a compound of formula (VIII), as defined hereinbefore, under the same conditions as the compounds of formula (VII), to yield compounds of formula (XVIII):

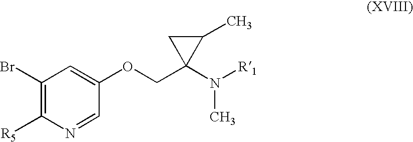

wherein R′₁ and R₅ are as defined hereinbefore,

the compounds of formulae (XVII) and (XVIII) constituting the compounds of formula (II/b) as defined hereinbefore.

According to another embodiment of the invention, compounds of formula (II), in the case where X represents an NR₆ group and R′₂ represents a tert-butoxycarbonyl group, of formula (II/c):

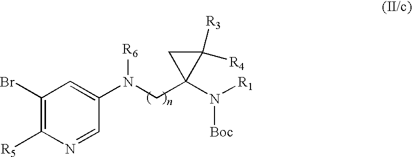

wherein R₁, R₃, R₄, R₅, R₆, Boc and n are as defined hereinbefore, may be prepared starting from a compound of formula (XI), as defined hereinbefore, which is placed in the presence of oxalyl chloride and DMSO to yield compounds of formula (XIX):

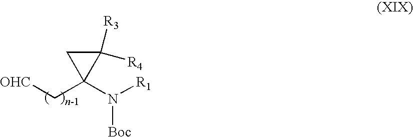

wherein R₁, R₃, R₄, Boc and n are as defined hereinbefore,

which compounds of formula (XIX) are reacted with a compound of formula (XX):

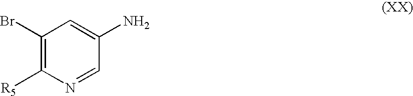

wherein R₅ is as defined hereinbefore, in the presence of acetic acid, then sodium cyanoborohydride, to yield compounds of formula (XXI):

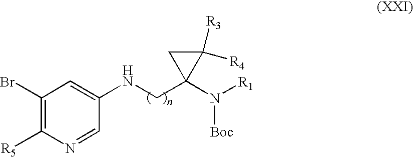

wherein R₁, R₃, R₄, R₅, Boc and n are as defined hereinbefore,

which compounds of formula (XXI) are:
either placed in the presence of formic acid and acetic anhydride to yield compounds of formula (XXII):

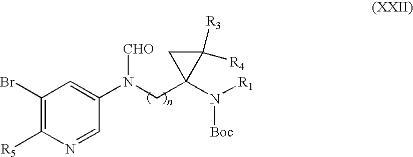

wherein R₁, R₃, R₄, R₅, Boc and n are as defined hereinbefore,

which compounds of formula (XXII) are placed in the presence of borane dimethyl sulphide complex to yield compounds of formula (XXIII):

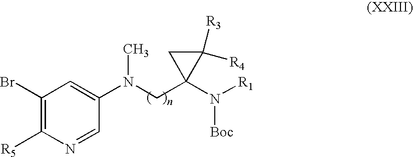

wherein R₁, R₃, R₄, R₅, Boc and n are as defined hereinbefore,
or reacted with a compound of formula (XXIV):

R′₆-L₆ (XXIV)

wherein R′₆ represents a linear or branched (C₁-C₆)alkyl group or an aryl-(C₁-C₆)alkyl group in which the alkyl moiety may be linear or branched and L₆ represents a leaving group customary in organic chemistry, in basic medium, to yield compounds of formula (XXV):

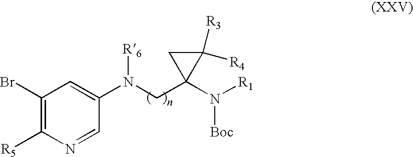

wherein R₁, R₃, R₄, R₅, R′₆, Boc and n are as defined hereinbefore,

the compounds of formulae (XXI), (XXIII) and (XXV) constituting the compounds of formula (II/c) as defined hereinbefore.

By virtue of their pharmacological properties as nicotinic ligands, and their selectivity for the receptor sub-type α4β2, the compounds of the present invention are of use in the treatment of deficiencies of memory associated with cerebral ageing and with neurodegenerative diseases such as Alzheimer's disease, Parkinson's disease, Pick's to disease, Korsakoff's disease and frontal lobe and subcortical dementias, and also for the treatment of mood disorders, Tourette's syndrome, attention-deficit hyperactivity syndrome, tobacco withdrawal and pain.

The present invention relates also to pharmaceutical compositions comprising as active ingredient at least one compound of formula (I), an isomer thereof, or an addition salt thereof with a pharmaceutically acceptable acid or base, alone or in combination with one or more pharmaceutically acceptable, inert, non-toxic excipients or carriers.

Pharmaceutical compositions according to the invention for parenteral injections include, especially, aqueous and non-aqueous sterile solutions, dispersions, suspensions and emulsions, and also sterile powders for reconstituting injectable solutions or dispersions.

Pharmaceutical compositions according to the invention for oral administration in solid form include, especially, tablets or dragées, sublingual tablets, sachets, capsules and granules and, for oral, nasal, buccal or ocular administration in liquid form, include, especially, emulsions, solutions, suspensions, drops, syrups and aerosols.

Pharmaceutical compositions for rectal or vaginal administration are preferably suppositories, and those for per- or trans-cutaneous administration include, especially, powders, aerosols, creams, ointments, gels and patches.

The pharmaceutical compositions mentioned hereinbefore illustrate the invention but do not limit it in any way.

Among the pharmaceutically acceptable, inert, non-toxic excipients or carriers there may be mentioned, by way of non-limiting example, diluents, solvents, preservatives, wetting agents, emulsifiers, dispersing agents, binders, swelling agents, disintegrating agents, retardants, lubricants, absorbents, suspending agents, colorants, flavourings etc.

The useful dosage varies according to the age and weight of the patient, the administration route and the pharmaceutical composition used, the nature and severity of the disorder and the administration of any associated treatments. The dosage ranges from 1 mg to 500 mg per day in one or more administrations.

The Examples that follow illustrate the invention but do not limit it in any way.

The starting materials used are products that are known or that are prepared according to known operating procedures. The various Preparations yield synthesis intermediates that are useful in the preparation of the compounds of the invention.

The structures of the compounds described in the Examples and Preparations were determined according to the usual spectrophotometric techniques (infrared, nuclear magnetic resonance, mass spectrometry, . . . ).

The melting points were determined using either a Kofler hot-plate, or a hot-plate under a microscope.

Preparation 1:

### tert-Butyl (1-{[(5-bromopyridin-3-yl)oxy]methyl}cyclopropyl)methylcarbamate

### Step 1: Methyl 1[(tert-butoxycarbonyl)amino]cyclopropanecarboxylate

A solution of 80 g of 1-(methoxycarbonyl)cyclopropanecarboxylic acid, 78 ml of triethylamine in 550 ml of toluene, to which 152 g of diphenylphosphoryl azide has been added, is heated to 80° C. Once the evolution of gas has ceased, the temperature is brought to 50° C. and 61 g of tert-butanol are added. After reaction for 7 hours at 80° C., the mixture is concentrated. The residue is taken up in ether, washed with saturated Na₂CO₃ solution and then with 1N hydrochloric acid solution, and subsequently with NaHCO₃ solution. After drying and removal of the organic phase by evaporation, the residue is taken up in 300 ml of cyclohexane and then concentrated to dryness. The residue obtained is triturated in pentane, filtered and then dried, allowing the expected product to be isolated.

### Step 2: Methyl 1[(tert-butoxycarbonyl)(methyl)amino]cyclopropanecarboxylate

24.7 g of 60% sodium hydride are added in portions to a solution, cooled to 5° C., of 99.7 g of the compound obtained in the above Step 1 in 1.7 1 of anhydrous dimethylformamide. After 15 minutes at 5° C. and then 3 hours at ambient temperature, 38.2 ml of methyl iodide are added dropwise. After reaction for 20 hours, the mixture is evaporated. The residue is taken up in ether and then treated in conventional manner. Chromatography on silica gel (dichloromethane) allows the expected product to be isolated.

### Step 3: tert-Butyl 1-(hydroxymethyl)cyclopropyl(methyl)carbamate

A 100 ml solution of 2M lithium borohydride in tetrahydrofuran is added to a solution of 23 g of the compound obtained in the above Step 2 in 100 ml of tetrahydrofuran. After stirring for 20 hours at ambient temperature, then for 8 hours at reflux, the reaction mixture is cooled to 0° C., hydrolysed, diluted with ether, decanted, dried and concentrated. Chromatography of the residue on silica gel (dichloromethane/tetrahydrofuran: 95/5) allows the expected product to be isolated.

### Step 4: fed-Butyl 1-(bromomethyl)cyclopropyl(methyl)carbamate

At 20° C., 7.9 g of triphenylphosphine and then 9.9 g of tetrabromomethane are added to a solution of 4 g of the compound obtained in the above Step 3 in 100 ml of ether. After stirring for 24 hours, filtering and concentrating to dryness, chromatography on silica gel (dichloromethane) allows the expected product to be isolated.

Melting point: 62-64° C.

### Step 5: tert-Butyl(1-{([(5-bromopyridin-3-yl)oxy]methyl}cyclopropyl)methylcarbamate

12.3 g of powdered potassium hydroxide are added to a solution of 13.1 g of 5-bromo-pyridin-3-ol in 375 ml of DMF. The reaction mixture is stirred for 40 minutes and then a solution of 24.3 g of the compound obtained in the above Step 4 in 115 ml of DMF is added in the course of 20 minutes. The whole is heated for 8 hours at 85° C. and then the DMF is evaporated off. The residue is taken up in aqueous 10% lithium chloride solution and extracted repeatedly with ethyl acetate, dried over sodium sulphate and then evaporated. Chromatography on silica gel (dichloromethane/tetrahydrofuran: 98/2) allows 23.9 g of the expected product to be obtained.

Preparation 2

### tert-Butyl(1-{[(5-bromo-6-chloropyridin-3-yl)oxy]methyl}cyclopropyl)methylcarbamate

9.5 g of caesium carbonate are added to a solution of 7.3 g of the compound obtained in Step 4 of Preparation 1 and 7.5 g of 5-bromo-6-chloropyridin-3-ol in 200 ml of 2-butanone. The reaction mixture is heated at reflux for 20 hours and then the butanone is evaporated off. The residue is taken up in saturated aqueous sodium carbonate solution and then extracted repeatedly with ether. The combined ethereal phases are then washed with saturated aqueous solutions of sodium carbonate and of sodium chloride and subsequently dried over sodium sulphate and concentrated to obtain 10.6 g of the expected product.

Preparation 3

### tert-Butyl(1-{[(5-bromo-6-methylpyridin-3-yl)oxy]methyl}cyclopropyl)methylcarbamate

13 g of caesium carbonate are added to a solution of 10.5 g of the compound obtained in Step 4 of Preparation 1 and 7.5 g of 5-bromo-6-methylpyridin-3-ol in 300 ml of 2-butanone. The reaction mixture is heated at reflux for 20 hours. After returning to ambient temperature, the minerals are filtered off and the butanone is evaporated off. Chromatography on silica gel (dichloromethane/butanone: 95/5) allows 14.8 g of the expected product to be obtained.

Preparation 4

### tert-Butyl(1-{[(5-bromo-6-fluoropyridin-3-yl)oxy]methyl}cyclopropyl)methylcarbamate

12.9 g of caesium carbonate are added to a solution of 10.5 g of the compound obtained in Step 4 of Preparation 1 and 5.8 g of 5-bromo-6-fluororopyridin-3-ol in 300 ml of butanone. The reaction mixture is heated for 20 hours at reflux and then filtered and concentrated. The residue is taken up in dichloromethane. After washing with saturated sodium chloride solution and drying over sodium sulphate, the organic phase is concentrated. Chromatography on silica gel (dichloromethane/butanone: 98/2) allows 10.7 g of the expected product to be obtained.

Preparation 5

### tert-Butyl(1-{[(5-bromo-6-chloropyridin-3-yl)oxy]methyl}cyclopropyl)carbamate

### Step 1: tert-Butyl 1-(hydroxymethyl)cyclopropylcarbamate

A 100 ml solution of 2M lithium borohydride in tetrahydrofuran is added to a solution of 23 g of the compound of Step 1 of Preparation 1 in 100 ml of tetrahydrofuran. After stirring for 20 hours at ambient temperature and then for 8 hours at reflux, the reaction mixture is cooled to 0° C., hydrolysed, diluted with ether, decanted, dried and concentrated. Chromatography of the residue on silica gel (dichloromethane/tetrahydrofuran: 95/5) allows the expected product to be isolated.

Melting point: 80-82° C.

### Step 2: tert-Butyl[1-(bromomethyl)cyclopropyl]carbamate

A solution of 92.5 g of carbon tetrabromide in 150 ml of ether is added at ambient temperature to a solution of 34.5 g of the compound of the above Step 1 and 73.5 g of triphenylphosphine in 750 ml of ether. After stirring for 20 hours, the reaction mixture is filtered and concentrated. Chromatography on silica gel (dichloromethane/cyclohexane: 50/50) allows 15 g of the expected product to be obtained.

### Step 3: tert-Butyl(1-{[(5-bromo-6-chloropyridin-3-yl)oxy]methyl}cyclopropyl)carbamate

The compound is obtained in accordance with the procedure of Preparation 3, using the compound of the above Step 2 and with the replacement of 5-bromo-6-methylpyridin-3-ol with 5-bromo-6-chloropyridin-3-ol.

Preparation 6

### (1S,2R),(1R,2S)-1-{[(5-Bromo-3-pyridinyl)oxy]methyl}-N,2-dimethylcyclopropanamine

### Step 1: Ethyl(1R,2S),(1S,2R)-1-isocyano-2-methylcyclopropanecarboxylate

A solution of 2.5 g of ethyl isocyanate, 2.3 cm³ of 1,2-dibromopropane, 25 cm³ of dimethyl sulphoxide and 60 cm³ of ether is added dropwise, in the course of one hour, to a suspension of 1.93 g of 60% sodium hydride in oil in 20 cm³ of ether. After heating at reflux for 2 hours, the reaction mixture is cooled and poured into a mixture of 50 cm³ of ice-water and 50 cm³ of ether. The aqueous phase is decanted off and extracted again with ether (3×40 cm³). The combined organic phases are washed with aqueous sodium chloride solution, dried over sodium sulphate and evaporated. Chromatography on silica gel (dichloromethane/tetrahydrofuran: 97/3) allows 4.88 g of the expected product to be obtained.

Diastereoisomeric ratio: 90/10.

### Step 2: [(1R,2S),(1S,2R)-2-Methyl-1-(methylamino)cyclopropyl]methanol

A solution of 4.88 g of the compound obtained in the above Step 1 in 85 cm³ of ether is added dropwise to a suspension of 3.73 g of lithium aluminium hydride in 250 cm³ of ether. The reaction mixture is heated at reflux for 4 hours and then stirred at ambient temperature for 16 hours. The reaction mixture is cooled in an ice-bath before the addition of sodium sulphate impregnated with water. After stirring for two hours, the minerals are filtered off, and the ethereal phase is dried over sodium sulphate and subsequently evaporated to obtain 2.75 g of the expected product.

Diastereoisomeric ratio: 90/10.

### Step 3: (1S,2R),(1R,2S)-1-{[(5-Bromo-3-pyridinyl)oxy]methyl}-N,2-dimethylcyclopropanamine

1.7 g of 60% sodium hydride in oil are added to 4.6 g of the compound obtained in the above Step 2 in 160 cm³ of dimethylformamide. The reaction mixture is stirred for one hour at ambient temperature and then 10.2 g of 3,5-dibromopyridine are added dropwise. The reaction mixture is heated for 16 hours at 60° C. and then the dimethylformamide is evaporated off. The residue is taken up in 300 cm³ of ether. The organic phase is washed with aqueous lithium chloride solution and then dried over sodium sulphate and concentrated. Chromatography on silica gel (dichloromethane/methanol: 96/4) allows 4.86 g of the expected compound to be obtained.

Mass spectrometry (ESI): m/z=271.1 Th ([M+H]⁺)

Preparation 7:

### tert-Butyl 1-(formyl)cyclopropyl(methyl)carbamate

At −60° C., 33.5 g of dimethyl sulphoxide are added in the course of 20 minutes to a solution containing 25.8 g of oxalyl chloride in 430 ml of dichloromethane. After stirring for 20 minutes at −60° C., a mixture containing 34.3 g of the compound of Step 3 of Preparation 1 in 100 ml of dichloromethane is added in the course of one hour at −60° C. After stirring for 30 minutes at −60° C., 81 ml of triethylamine are added in the course of 20 minutes at −60° C. and then the temperature is allowed to return to 20° C. 60 ml of water are added and the aqueous phase is decanted off and extracted repeatedly with dichloromethane. The combined dichloromethane phases are washed with saturated sodium chloride solution and dried over sodium sulphate and then concentrated to dryness. Chromatography on silica gel (dichloromethane/tetrahydrofuran: 97/3) allows 31.2 g of the expected product to be obtained.

Preparation 8

### tert-Butyl(1-{[(5-bromopyridin-3-yl)amino]methyl}cyclopropyl)methylcarbamate

6 ml of acetic acid are added to a solution containing 6 g of the compound of Preparation 7 and 5.2 g (0.03 mol) of 3-amino-5-bromopyridine in 60 ml of methanol. Stirring is carried out for 30 minutes at ambient temperature. Cooling to 5° C. is carried out and 2.44 g of sodium cyanoborohydride are added in portions. Stirring is carried out for 4 days at ambient temperature. 6.3 ml of water are added and concentration to dryness is carried out. The residue is taken up in 30 ml of saturated potassium carbonate solution in water and extraction is carried out with dichloromethane. The filtrate is dried over sodium sulphate and then concentrated. Chromatography on silica gel (dichloromethane/butanone: 90/10) allows 7.6 g of the expected product to be obtained.

Melting point (cap): 96° C.

Preparation 9

### tert-Butyl(1-{[(5-bromopyridin-3-yl)(methyl)amino]methyl}cyclopropyl)methylcarbamate

Step 1: tert-Butyl(1-{[(5-bromopyridin-3-yl)(formyl)amino]methyl}cyclopropyl)-methylcarbamate

2.69 ml of formic acid in 5.38 ml of acetic anhydride are added at 0° C. in the course of 20 minutes to 7.64 g of the compound of Preparation 8. The mixture is heated at 50° C. for 2 hours. It is allowed to cool to 20° C. and 5.38 ml of tetrahydrofuran are added. Cooling to −20° C. is carried out. 7.64 g of the compound of Preparation 8 dissolved in 11 ml of tetrahydrofuran are added. Stirring is carried out for 1 hour at −20° C. and the mixture is then maintained at 0° C. for 20 hours. Concentration to dryness is carried out and the residue is taken up in dichloromethane. The whole is washed twice with aqueous 10% sodium carbonate solution, dried over sodium sulphate and concentrated to dryness. Chromatography on silica gel (dichloromethane/butanone: 90/10) allows 8.09 g of the expected product to be obtained.

### Step 2: tert-Butyl(1-{[(5-bromopyridin-3-yl)(methyl)amino]methyl}cyclopropyl)-methylcarbamate

At 0° C., 5 ml of borane dimethyl sulphide complex (BMS) are poured into a solution of 7.7 g (0.02 mol) of the product obtained in the above Step 1 in 80 ml of tetrahydrofuran. The temperature is allowed to rise to 20° C. and heating at reflux is then carried out for 3 hours. Cooling to 0° C. is carried out, and then 10 ml of methanol are added dropwise. Concentration to dryness is carried out and the residue is taken up in dichloromethane, washed with aqueous 10% sodium carbonate solution, dried over sodium sulphate and concentrated to dryness. Chromatography on silica gel (dichloromethane) allows 5.68 g of the expected product to be obtained.

Preparation 10

### tert-Butyl(1-{[(5-Bromo-6-chloropyridin-3-yl)amino]methyl}cyclopropyl)-methylcarbamate

The compound is obtained in accordance with the procedure of Preparation 8, with the replacement of 3-amino-5-bromopyridine with 3-amino-5-bromo-6-chloropyridine.

Preparation 11

### tert-Butyl(1-{[(5-bromo-6-chloropyridin-3-yl)(methyl)amino]methyl}cyclopropyl)-methylcarbamate

### Step 1: tert-Butyl(1-{[(5-bromo-6-chloropyridin-3-yl)(formyl)amino]methyl}-cyclopropyl)methylcarbamate

The compound is obtained in accordance with the procedure of Step 1 of Preparation 9, with the replacement of the compound of Preparation 8 with the compound of Preparation 10.

### Step 2: tert-Butyl(1-{[(5-bromo-6-chloropyridin-3-yl)(methyl)amino]-methyl}cyclopropyl)methylcarbamate

The compound is obtained in accordance with the procedure of Step 2 of Preparation 9, using the compound obtained in the above Step 1.

Preparation 2

### tert-Butyl(1-{[(5-bromo-6-methylpyridin-3-yl)amino]methyl}cyclopropyl)methylcarbamate

The compound is obtained in accordance with the procedure of Preparation 8, with the replacement of 3-amino-5-bromopyridine with 3-amino-5-bromo-6-methylpyridine.

Preparation 13

### tert-Butyl(1-{[(5-bromo-6-methylpyridin-3-yl)methyl)amino]methyl}cyclopropyl)-methylcarbamate

### Step 1: tert-Butyl(1-{[(5-bromo-6-methylpyridin-3-yl)(formyl)amino]methyl}-cyclopropyl)methylcarbamate

The compound is obtained in accordance with the procedure of Step 1 of Preparation 9, with the replacement of the compound of Preparation 8 with the compound of Preparation 12.

### Step 2: tert-Butyl(1-{[(5-bromo-6-methylpyridin-3-yl)methyl)amino]methyl}-cyclopropyl)methylcarbamate

The compound is obtained in accordance with the procedure of Step 2 of Preparation 9, using the compound obtained in the above Step 1.

### EXAMPLE 1

### [1-({[5-(3-Methoxyphenyl)pyridin-3-yl]oxy}methyl)cyclopropyl]methylamine dihydrochloride

### Step 1: tert-Butyl[1-({[5-(3-methoxyphenyl)pyridin-3-yl]oxy}methyl)cyclopropyl]-methylcarbamate

1 g of tetrakis(triphenylphosphine)palladium are added under nitrogen to a solution of 6.3 g of the compound of Preparation 1 in 120 ml of toluene. The mixture is stirred for 20 minutes and then a solution of 4.09 g of (3-methoxyphenyl)boronic acid in 110 ml of ethanol and 60 ml of saturated aqueous sodium hydrogen carbonate solution are added. The reaction mixture is heated for 4 hours at 80° C. and then filtered and decanted. The organic phase is washed with 10% sodium hydrogen carbonate solution and then with 10% sodium chloride solution and subsequently dried over sodium sulphate and concentrated. Chromatography of the residue on silica gel (dichloromethane/tetrahydrofuran: 97/3) allows 5.06 g of the expected product to be obtained.

### Step 2: [1-({[5-(3-Methoxyphenyl)pyridin-3-yl]oxy}methyl)cyclopropyl]methylamine dihydrochloride

50 ml of 4N hydrochloric acid solution in dioxane are added to a solution of 5 g of the compound obtained in the above Step 1 in 25 ml of dioxane. The mixture is stirred for 20 hours, diluted with ether and then filtered to obtain 4.6 g of the desired product.

Melting point (cap): 210-212° C.

Mass spectrometry (ESI) m/z=285.1582 Th ([M+H]⁺)

### EXAMPLE 2

### Methyl[1-({[5-(4-methylphenyl)pyridin-3-yl]oxy}methyl)cyclopropyl]amine dihydrochloride

### Step 1: tert-Butyl methyl[1-({[5-(4-methylphenyl)pyridin-3-yl]oxy}methyl)cyclopropyl]-carbamate

The compound is obtained in accordance with the procedure of Step 1 of Example 1, with the replacement of (3-methoxyphenyl)boronic acid with (4-methylphenyl)boronic acid.

### Step 2: Methyl[1-({[5-(4-methylphenyl)pyridin-3-yl]oxy}methyl)cyclopropyl]amine dihydrochloride

10 ml of 4N hydrochloric acid solution in dioxane are added to a solution of 0.78 g of the compound obtained in the above Step 1 in 5 ml of dioxane. The mixture is stirred for 20 hours and then the solvent is evaporated off. The residue is dissolved in ethanol and the ethanol is then evaporated off. The crystallised product is stirred in the presence of ether and then filtered and dried to obtain 0.7 g (98%) of the expected product.

Melting point (cap): 218-223° C.

Mass spectrometry (ESI) m/z=269.1643 Th ([M+H]⁺)

### EXAMPLE 3

### [1-({[5-(4-Methoxyphenyl)pyridin-3-yl]oxy}methyl)cyclopropyl]methylamine dihydrochloride

### Step 1: tert-Butyl[1-({[5-(4-methoxyphenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (4-methoxyphenyl)boronic acid.

### Step 2: [1-({[5-(4-Methoxyphenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 215-220° C.

Mass spectrometry (ESI) m/z=285.1585 Th ([M+H]⁺)

### EXAMPLE 4

### Methyl(1-{[(5-phenylpyridin-3-yl)oxy]methyl{cyclopropyl)amine dihyhdrochloride

### Step 1: tert-Butyl methyl(1-{[(5-phenylpyridin-3-yl)oxy]methyl}cyclopropyl)carbamate

The compound is obtained in accordance with the procedure of Step 1 of Example 1, with the replacement of (3-methoxyphenyl)boronic acid with phenylboronic acid.

### Step 2: Methyl(1-{[(5-phenylpyridin-3-yl)oxy]methyl}cyclopropyl)amine dihydrochloride

The compound is obtained in accordance with the procedure of Step 2 of Example 2, using the compound obtained in the above Step 1.

Melting point (cap): 205-210° C.

Mass spectrometry (ESI) m/z=255.1481 Th ([M+H]⁺)

### EXAMPLE 5

### [1-({[(5-(4-Fluorophenyl)pyridin-3-yl]oxy}methyl)cyclopropyl]methylamine dihydrochloride

### Step 1: tert-Butyl[1-({[5-(4-fluorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (4-fluorophenyl)boronic acid.

### Step 2: [1-({[5-(4-Fluorophenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 218-223° C.

Mass spectrometry (ESI) m/z=273.1402 Th ([M+H]⁺)

### EXAMPLE 6

### [1-({[5-(4-Nitrophenyl)pyridin-3-yl]oxy}methyl)cyclopropyl]methylamine dihydrochloride

### Step 1: tert-Butyl[1-({[5-(4-nitrophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (4-nitrophenyl)boronic acid.

### Step 2: [1-({[5-(4-Nitrophenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 212-217° C.

Mass spectrometry (ESI) m/z=300.1340 Th ([M+H]⁺)

### EXAMPLE 7

### [1-({[5-(4-Chlorophenyl)pyridin-3-yl]oxy}methyl)cyclopropyl]methylamine dihydrochloride

### Step 1: tert-Butyl[1-({[5-(4-chlorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (4-chlorophenyl)boronic acid.

### Step 2: [1-({[5-(4-Fluorophenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 208-213° C.

Mass spectrometry (ESI) m/z=289.1108 Th ([M+H]⁺)

### EXAMPLE 8

### [1-({[6-Chloro-5-(3-methoxyphenyl)pyridin-3-yl]oxy}methyl)cyclopropyl]-methylamine dihydrochloride

### Step 1: tert-Butyl[1-({[6-chloro-5-(3-methoxyphenyl)pyridin-3-yl]oxy}methyl)-cyclopropyl]methylcarbamate

The compound is obtained in accordance with the procedure of Step 1 of Example 1, using the compound of Preparation 2 instead of the compound of Preparation 1.

### Step 2: [1-({[6-Chloro-5-(3-methoxyphenyl)pyridin-3-yl]oxy}methyl)-cyclopropyl]methylamine dihydrochloride

The compound is obtained in accordance with the procedure of Step 2 of Example 2, using the compound obtained in the above Step 1.

Mass spectrometry (ESI) m/z=319.1219 Th ([M+H]⁺)

### EXAMPLE 9

### (1-{[(6-Chloro-5-phenylpyridin-3-yl)oxy]methyl}cyclopropyl)methylamine dihydrochloride

### Step 1: tert-Butyl(1-{[(6-chloro-5-phenylpyridin-3-yl)oxy]methyl}cyclopropyl)-methylcarbamate

The compound is obtained in accordance with the procedure of Step 1 of Example 1, using the compound of Preparation 2 instead of the compound of Preparation 1 and with the replacement of (3-methoxyphenyl)boronic acid with phenylboronic acid.

### Step 2: (1-{[(6-Chloro-5-phenylpyridin-3-yl)oxy]methyl}cyclopropyl]methylamine dihydrochloride

The compound is obtained in accordance with the procedure of Step 2 of Example 2, using the compound obtained in the above Step 1.

Melting point (cap): 130-135° C.

Mass spectrometry (ESI) m/z=289.1094 Th ([M+H]⁺)

### EXAMPLE 10

### [1-({[6-Chloro-5-(4-methylphenyl)pyridin-3-yl]oxy}methyl)cyclopropyl]methylamine dihydrochloride

### Step 1: tert-Butyl[1-({[6-chloro-5-(4-methylphenyl)pyridin-3-yl]oxy}methyl)cyclopropyl]methylcarbamate

The compound is obtained in accordance with the procedure of Step 1 of Example 1, using the compound of Preparation 2 instead of the compound of Preparation 1 and with the replacement of (3-methoxyphenyl)boronic acid with (4-methylphenyl)boronic acid.

### Step 2: [1-({[6-Chloro-5-(4-methylphenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 150-155° C.

Mass spectrometry (ESI) m/z=303.1249 Th ([M+H]⁺)

### EXAMPLE 11

### [1-({[5-(4-Methoxyphenyl)pyridin-3-yl]oxy}methyl)cyclopropyl]methylamine dihydrochloride

### Step 1: tert-Butyl[1-({[6-chloro-5-(4-methoxyphenyl)pyridin-3-yl]oxy}methyl)cyclopropyl]methylcarbamate

The compound is obtained in accordance with the procedure of Step 1 of Example 1, using the compound of Preparation 2 instead of the compound of Preparation 1 and with the replacement of (3-methoxyphenyl)boronic acid with (4-methoxyphenyl)boronic acid.

### Step 2: 1-({[5-(4-Methoxyphenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 180-185° C.

Mass spectrometry (ESI) m/z=319.1199 Th ([M+H]⁺)

### EXAMPLE 12

### [1-({[6-Chloro-5-(4-nitrophenyl)pyridin-3-yl]oxy}methyl)cyclopropyl]methylamine hydrochloride

### Step 1: tert-Butyl[1-({[6-chloro-5-(4-nitrophenyl)pyridin-3-yl]oxy}methyl)cyclopropyl]methylcarbamate

The compound is obtained in accordance with the procedure of Step 1 of Example 1 by reacting 1.1 g of the compound of Preparation 2 and 0.7 g of (4-nitrophenyl)boronic acid. Chromatography on silica gel (dichloromethane/tetrahydrofuran: 97/3) allows 0.63 g of the expected product to be obtained.

### Step 2: [1-({[6-Chloro-5-(4-nitrophenyl)pyridin-3-yl]oxy}methyl)cyclopropyl]-methylamine hydrochloride

The compound is obtained in accordance with the procedure of Step 2 of Example 2, using the compound obtained in the above Step 1.

Melting point (cap): 145-150° C.

Mass spectrometry (ESI) m/z=334.0945 Th ([M+H]⁺)

### EXAMPLE 13

### [1-({[5-(4-Chlorophenyl)pyridin-3-yl]oxy}methyl)cyclopropyl]methylamine hydrochloride

### Step 1: tert-Butyl[4({[6-chloro-5-(4-chlorophenyl)pyridin-3-yl]oxy}methyl)cyclopropyl]methylcarbamate

The compound is obtained in accordance with the procedure of Step 1 of Example 12, with the replacement of (4-nitrophenyl)boronic acid with (4-chlorophenyl)boronic acid.

### Step 2: [1-({[5-(4-Chlorophenyl)pyridin-3-yl]oxy}methyl)cyclopropyl]methylamine hydrochloride

The compound is obtained in accordance with the procedure of Step 2 of Example 2, using the compound obtained in the above Step 1.

Melting point (cap): 160-165° C.

Mass spectrometry (ESI) m/z=323.0699 Th ([M+H]⁺)

### EXAMPLE 14

### [4-(2-Chloro-5-{[1-(methylamino)cyclopropyl]methoxy}pyridin-3-yl)phenyl]methanol dihydrochloride

### Step 1: tert-Butyl[1-[({6-chloro-5-(4-(hydroxymethyl)phenyl]pyridin-3-yl]oxy)methyl-cyclopropyl}methylcarbamate

The compound is obtained in accordance with the procedure of Step 1 of Example 1, using the compound of Preparation 2 instead of the compound of Preparation 1 and with the replacement of (3-methoxyphenyl)boronic acid with (4-hydroxymethylphenyl)boronic acid.

### Step 2: [4-(2-Chloro-5-{[1-(methylamino)cyclopropyl]methoxy}pyridin-3-yl)phenyl]methanol dihydrochloride

The compound is obtained in accordance with the procedure of Step 2 of Example 1, using the compound obtained in the above Step 1.

Melting point (cap): 142-146° C.

Mass spectrometry (ESI) m/z=319 Th ([M+H]⁺)

### EXAMPLE 15

### [1-({[5-(4-Chlorophenyl)-6-methylpyridin-3-yl]oxy}methyl)cyclopropyl]methylamine dihydrochloride

### Step 1: tert-Butyl[1-({[5-(4-chlorophenyl)-6-methylpyridin-3-yl]oxy}methyl)cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (4-chlorophenyl)boronic acid.

### Step 2: [1-({[5-(4-Chlorophenyl)-6-methylpyridin-3-yl]oxy}methyl)cyclopropyl]-methylamine dihydrochloride

The compound is obtained in accordance with the procedure of Step 2 of Example 2, using the compound obtained in the above Step 1.

Melting point (cap): 194-199° C.

Mass spectrometry (ESI) m/z =303.1 Th ([M+H]⁺)

### EXAMPLE 16

### [1-({[5-(3-Chlorophenyl)-6-methylpyridin-3-yl]oxy}methyl)cyclopropyl]methylamine dihydrochloride

### Step 1: tert-Butyl 1-({[5-(3-chlorophenyl)-6-methylpyridin-3-yl]oxy}methyl)-cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (3-chlorophenyl)boronic acid.

### Step 2: [1-({[5-(3-Chlorophenyl)-6-methylpyridin-3-yl]oxy}methyl)cyclopropyl]-methylamine dihydrochloride

The compound is obtained in accordance with the procedure of Step 2 of Example 2, using the compound obtained in the above Step 1.

Melting point (cap): 223-227° C.

Mass spectrometry (ESI) m/z=303.1 Th ([M+H]⁺)

### EXAMPLE 17

### 3-(2-Methyl-5-{[1-(methylamino)cyclopropyl]methoxy}pyridin-3-yl)benzonitrile dihydrochloride

### Step 1: tert-Butyl[1-({[5-(3-cyanophenyl)-6-methylpyridin-3-yl]oxy}methyl)cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (3-cyanophenyl)boronic acid.

### Step 2: 3-(2-Methyl-5-{[1-(methylamino)cyclopropyl]methoxy}pyridin-3-yl)benzonitrile dihydrochloride

The compound is obtained in accordance with the procedure of Step 2 of Example 2, using the compound obtained in the above Step 1.

Melting point (cap): 238-242° C.

Mass spectrometry (ESI) m/z=294.2 Th ([M+H]⁺)

### EXAMPLE 18

### 4-(2-Chloro-5-{[1-(methylamino)cyclopropyl]methoxy}pyridin-3-yl)benzonitrile hydrochloride

### Step 1: tert-Butyl 1-({[6-chloro-5-(4-cyanophenyl)pyridin-3-yl]oxy}methyl)cyclopropyl]methylcarbamate

The compound is obtained in accordance with the procedure of Step 1 of Example 12, with the replacement of (4-nitrophenyl)boronic acid with (4-cyanophenyl)boronic acid.

### Step 2: 4-(2-Chloro-5-{[1-(methylamino)cyclopropyl]methoxy}pyridin-3-yl)benzonitrile hydrochloride

8 ml of 4N hydrochloric acid solution in dioxane are added to a solution of 0.6 g of the compound obtained in the above Step 1 in 100 ml of acetonitrile. After stirring for 16 hours, the reaction mixture is diluted with ether and the precipitate is filtered off. The precipitate is dissolved in ethanol, the solution is concentrated and then the residue obtained is dissolved again in ethanol. After dilution with ether and filtration, 0.53 g of the expected compound is obtained.

Melting point (cap): 215-220° C.

Mass spectrometry (ESI) m/z=314 Th ([M+H]⁺)

### EXAMPLE 19

### 4-(2-Methyl-5-{[1-(methylamino)cyclopropyl]methoxy}pyridin-3-yl)benzonitrile dihydrochloride

### Step 1: tert-Butyl[1-({[5-(4-cyanophenyl)-6-methylpyridin-3-yl]oxy}methyl)cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (4-cyanophenyl)boronic acid.

### Step 2: 4-(2-Methyl-5-{[1-(methylamino)cyclopropyl]methoxy}pyridin-3-yl)benzonitrile dihydrochloride

The compound is obtained in accordance with the procedure of Step 2 of Example 18, using the compound obtained in the above Step 1.

Melting point (cap): 220-224° C.

Mass spectrometry (ESI) m/z=294.1622 Th ([M+H]⁺)

### EXAMPLE 20

### 4-(5-({[1-(Methylamino)cyclopropyl]methoxy}pyridin-3-yl)benzonitrile dihydrochloride

### Step 1: tert-Butyl[1-({[5-(4-cyanophenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (4-cyanophenyl)boronic acid.

### Step 2: 4-(5-{[1-(Methylamino)cyclopropyl]methoxy}pyridin-3-yl)benzonitrile dihydrochloride

7.5 ml of trifluoroacetic acid are added to a solution of 0.85 g of the compound obtained in the above Step 1 in 7.5 ml of dichloromethane. After stirring for 20 hours, the reaction mixture is concentrated to dryness and taken up in a mixture of dichloromethane and saturated aqueous sodium carbonate solution. The organic phase is decanted off, dried over sodium sulphate and then concentrated. Chromatography of the residue on silica gel (toluene/ethanol: 93/7) allows the base of the desired compound to be isolated. Following dissolution of the base in ether, addition of hydrochloric acid solution in ether, filtration and drying, 0.52 g of the expected product is obtained.

Melting point (cap): 150-160° C.

Mass spectrometry (ESI) m/z=280.1439 Th ([M+H]⁺)

### EXAMPLE 21

### 3-(5-{[1-(Methylamino)cyclopropyl]methoxy}pyridin-3-yl)benzonitrile dihydrochloride

### Step 1: tert-Butyl[1-({[5-(3-cyanophenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (3-cyanophenyl)boronic acid.

### Step 2: 3-(5-{[1-(Methylamino)cyclopropyl]methoxy}pyridin-3-yl)benzonitrile dihydrochloride

The compound is obtained in accordance with the procedure of Step 2 of Example 20, using the compound obtained in the above Step 1.

Melting point (cap): 110-120° C.

Mass spectrometry (ESI) m/z=280.1435 Th ([M+H]⁺)

### EXAMPLE 22

### [1-({[5-(4-Chlorophenyl)-6-fluoropyridin-3-yl]oxy}methyl)cyclopropyl]methylamine hydrochloride

### Step 1: tert-Butyl[1-({[5-(4-chlorophenyl)-6-fluoropyridin-3-yl]oxy}methyl)cyclopropyl]methylcarbamate

The compound is obtained in accordance with the procedure of Step 1 of Example 1, using the compound of Preparation 4 instead of the compound of Preparation 1 and with the replacement of (3-methoxyphenyl)boronic acid with (4-chlorophenyl)boronic acid.

### Step 2: [1-({[5-(4-Chlorophenyl)-6-fluoropyridin-3-yl]oxy}methyl)cyclopropyl]-methylamine hydrochloride

2.5 ml of trifluoroacetic acid are added to a solution of 0.4 g of the compound of the above Step 1 in 5 ml of dichloromethane. After stirring for 20 hours, the reaction mixture is concentrated to dryness and taken up in a mixture of dichloromethane and saturated aqueous sodium carbonate solution. The organic phase is decanted off, dried over sodium sulphate and then concentrated. The base obtained is taken up in ethanol, and the hydrochloride is precipitated by addition of hydrochloric acid solution in ether and then dilution with ether. After filtration and drying, 0.24 g of the expected product is obtained.

Melting point (cap): 55-60° C.

Mass spectrometry (ESI) m/z=307.1043 Th ([M+H]⁺)

### EXAMPLE 23

### [1-({[5-(3-Chlorophenyl)-6-fluoropyridin-3-yl]oxy}methyl)cyclopropyl]methylamine hydrochloride

### Step 1: tert-Butyl[1-({[5-(3-chlorophenyl)-6-fluoropyridin-3-yl]oxy}methyl)cyclopropyl]methylcarbamate

The compound is obtained in accordance with the procedure of Step 1 of Example 1, using the compound of Preparation 4 instead of the compound of Preparation 1 and with the replacement of (3-methoxyphenyl)boronic acid with (3-chlorophenyl)boronic acid.

### Step 2: [1-({[5-(3-Chlorophenyl)-6-fluoropyridin-3-yl]oxy}methyl)cyclopropyl]-methylamine dihydrochloride

The compound is obtained in accordance with the procedure of Step 2 of Example 22, using the compound obtained in the above Step 1.

Melting point (cap): 126-130° C.

Mass spectrometry (ESI) m/z=307.1018 Th ([M+H]³⁰ )

### EXAMPLE 24

### 4-(2-Fluoro-5-{[1-(methylamino)cyclopropyl]methoxy}pyridin-3-yl)benzonitrile hydrochloride

### Step 1: tert-Butyl[1-({[6-fluoro-5-(4-cyanophenyl)pyridin-3-yl]oxy}methyl)cyclopropyl]methylcarbamate

The compound is obtained in accordance with the procedure of Step 1 of Example 1, using the compound of Preparation 4 instead of the compound of Preparation 1 and with the replacement of (3-methoxyphenyl)boronic acid with (4-cyanophenyl)boronic acid.

### Step 2: 4-(2-Fluoro-5-{[1-(methylamino)cyclopropyl]methoxy}pyridin-3-yl)benzonitrile hydrochloride

The compound is obtained in accordance with the procedure of Step 2 of Example 22, using the compound obtained in the above Step 1.

Melting point (cap): 90-105° C.

Mass spectrometry (ESI) m/z=298.1370 Th ([M+H]⁺)

### EXAMPLE 25

### 3-(2-Fluoro-5-{[1-(methylamino)cyclopropyl]methoxy}pyridin-3-yl)benzonitrile hydrochloride

### Step 1: tert-Butyl[1-({[6-fluoro-5-(3-cyanophenyl)pyridin-3-yl]oxy}methyl)cyclopropyl]methylcarbamate

The compound is obtained in accordance with the procedure of Step 1 of Example 1, using the compound of Preparation 4 instead of the compound of Preparation 1 and with the replacement of (3-methoxyphenyl)boronic acid with (3-cyanophenyl)boronic acid.

### Step 2: 3-(2-Fluoro-5-{[1-(methylamino)cyclopropyl]methoxy}pyridin-3-yl)benzonitrile hydrochloride

The compound is obtained in accordance with the procedure of Step 2 of Example 22, using the compound obtained in the above Step 1.

Melting point (cap): 211-215° C.

Mass spectrometry (ESI) m/z=298.1363 Th ([M+H]⁺)

### EXAMPLE 26

### [1-({[6-Chloro-5-(4-fluorophenyl)pyridin-3-yl]oxy}methyl)cyclopropyl]methylamine hydrochloride

0.16 g of tetrakis(triphenylphosphine)palladium (Pd(PPh₃)₄) are added, under argon, to a solution of 1.63 g of the compound of Preparation 2 in 30 ml of toluene. The mixture is stirred for 45 minutes and then a solution of 0.59 g of (4-fluorophenyl)boronic acid in 15 ml of ethanol and 15 ml of saturated aqueous sodium hydrogen carbonate solution are added. The reaction mixture is heated for 4 hours 30 minutes at 85° C. and then filtered and decanted. The organic phase is dried over sodium sulphate and concentrated to obtain 1.95 g of crude coupling product. The crude product is dissolved in 15 ml of dichloromethane and then 3.5 ml of trifluoroacetic acid are added. After stirring for 20 hours, deprotection is complete and the reaction mixture is concentrated. The residue obtained is chromatographed on an RP18 column 12-25μ (water/trifluoroacetic acid: 1000/2.5 to water/acetonitrile/trifluoroacetic acid: 750/250/2.5). The chromatography fractions are analysed and combined, and then the acetonitrile is evaporated off. The residual aqueous solution is neutralised and then saturated with solid sodium hydrogen carbonate and subsequently extracted with ethyl acetate. After drying over sodium sulphate and concentrating the organic phase, the base obtained is dissolved in ethanol and 1.5 ml of 4N hydrochloric acid solution in dioxane is added. After concentration and crystallisation there is obtained, after washing with ether and drying, 0.86 g of the expected product.

Melting point (cap): 194-196° C.

Mass spectrometry (ESI) m/z=307.0998 Th ([M+H]⁺)

### EXAMPLE 27

### [1-({[5-(3-Aminophenyl)-6-chloropyridin-3-yl]oxy}methyl)cyclopropyl]methylamine dihydrochloride

The compound is obtained in accordance with the procedure of Example 26, with the replacement of (4-fluorophenyl)boronic acid with (3-aminophenyl)boronic acid.

Mass spectrometry (ESI) m/z=304.1146 Th ([M+]⁺)

### EXAMPLE 28

### [1-({[6-Chloro-5-(3-nitrophenyl)pyridin-3-yl]oxy}methyl)cyclopropyl]methylamine hydrochloride

The compound is obtained in accordance with the procedure of Example 26, with the replacement of (4-fluorophenyl)boronic acid with (3-nitrophenyl)boronic acid.

Melting point (cap): 229-232° C.

Mass spectrometry (ESI) m/z=334.0923 Th ([M+H]⁺)

### EXAMPLE 29

### {1-[({6-Chloro-5-[4-(methylthio)phenyl]pyridin-3-yl}oxy)methyl]cyclopropyl}-methylamine dihydrochloride

The compound is obtained in accordance with the procedure of Example 26, with the replacement of (4-fluorophenyl)boronic acid with (4-methylthiophenyl)boronic acid.

Melting point (cap): 144-148° C.

Mass spectrometry (ESI) m/z=335.0952 Th ([M+H]⁺)

### EXAMPLE 30

### [1-({[6-Chloro-5-(4-ethylphenyl)pyridin-3-yl]oxy}methyl)cyclopropyl]methylamine dihydrochloride

The compound is obtained in accordance with the procedure of Example 26, with the replacement of (4-fluorophenyl)boronic acid with (4-ethylphenyl)boronic acid.

Melting point (cap): 112-114° C.

Mass spectrometry (ESI) m/z=317.1382 Th ([M+H]⁺)

### EXAMPLE 31

### [1-({[6-Chloro-5-(2-methylphenyl)pyridin-3-yl]oxy}methyl)cyclopropyl]methylamine hydrochloride

The compound is obtained in accordance with the procedure of Example 26, with the replacement of (4-fluorophenyl)boronic acid with (2-methylphenyl)boronic acid.

Melting point (cap): 130-132° C.

Mass spectrometry (ESI) m/z=303.1326 Th ([M+H]⁺)

### EXAMPLE 32

### [1-({[6-Chloro-5-(3-fluorophenyl)pyridin-3-yl]oxy}methyl)cyclopropyl]methylamine hydrochloride

The compound is obtained in accordance with the procedure of Example 26, with the replacement of (4-fluorophenyl)boronic acid with (3-fluorophenyl)boronic acid.

Melting point (cap): 172-175° C.

Mass spectrometry (ESI) m/z=307.0976 Th ([M+H]⁺)

### EXAMPLE 33

### [1-({[6-Chloro-5-(3-methylphenyl)pyridin-3-yl]oxy}methyl)cyclopropyl]methylamine hydrochloride

The compound is obtained in accordance with the procedure of Example 26, with the replacement of (4-fluorophenyl)boronic acid with (3-methylphenyl)boronic acid.

Melting point (cap): 152-154° C.

Mass spectrometry (ESI) m/z=303.1239 Th ([M+H]⁺)

### EXAMPLE 34

### [1-({[6-Chloro-5-(3-chlorophenyl)pyridin-3-yl]oxy}methyl)cyclopropyl]methylamine hydrochloride

The compound is obtained in accordance with the procedure of Example 26, with the replacement of (4-fluorophenyl)boronic acid with (3-chlorophenyl)boronic acid.

Melting point (cap): 182-184° C.

Mass spectrometry (ESI) m/z=323.0724 Th ([M+H]⁺)

### EXAMPLE 35

### 3-(2-Chloro-5-{[1-(methylamino)cyclopropyl]methoxy}pyridin-3-yl)benzonitrile hydrochloride

The compound is obtained in accordance with the procedure of Example 26, with the replacement of (4-fluorophenyl)boronic acid with (3-cyanophenyl)boronic acid.

Melting point (cap): 228-232° C.

Mass spectrometry (ESI) m/z=314.1034 Th ([M+H]⁺)

### EXAMPLE 36

### 1-({[6-Chloro-5-(2,3,4-trimethoxyphenyl)pyridin-3-yl]oxy}methyl)cyclopropyl]methylamine hydrochloride

The compound is obtained in accordance with the procedure of Example 26, with the replacement of (4-fluorophenyl)boronic acid with (2,3,4-trimethoxyphenyl)boronic acid.

Melting point (cap): 168-170° C.

Mass spectrometry (ESI) m/z=379.1433 Th ([M+H]⁺)

### EXAMPLE 37

### [1-({[6-Chloro-5-(3,4,5-trimethoxyphenyl)pyridin-3-yl]oxy}methyl)cyclopropyl]methylamine hydrochloride

The compound is obtained in accordance with the procedure of Example 26, with the replacement of (4-fluorophenyl)boronic acid with (3,4,5-trimethoxyphenyl)boronic acid.

Melting point (cap): 138-140° C.

Mass spectrometry (ESI) m/z=379.1436 Th ([M+H]⁺)

### EXAMPLE 38

### {1-[({5-[3,5-bis(Trifluoromethyl)phenyl]-6-chloropyridin-3-yl}oxy)methyl]cyclopropyl}methylamine hydrochloride

The compound is obtained in accordance with the procedure of Example 26, with the replacement of (4-fluorophenyl)boronic acid with [3,5-bis(trifluoromethyl)phenyl)]boronic acid.

Melting point (cap): 182-188° C.

Mass spectrometry (ESI) m/z=425.0875 Th ([M+H]⁺)

### EXAMPLE 39

### [1-({[6-Chloro-5-(2,5-difluorophenyl)pyridin-3-yl]oxy}methyl)cyclopropyl]-methylamine hydrochloride

The compound is obtained in accordance with the procedure of Example 26, with the replacement of (4-fluorophenyl)boronic acid with (2,5-difluorophenyl)boronic acid.

Melting point (cap): 140-142° C.

Mass spectrometry (ESI) m/z=325.0887 Th ([M+H]⁺)

### EXAMPLE 40

### [1-({[6-Chloro-5-(2,5-dichlorophenyl)pyridin-3-yl]oxy}methyl)cyclopropyl]-methylamine hydrochloride

The compound is obtained in accordance with the procedure of Example 26, with the replacement of (4-fluorophenyl)boronic acid with (2,5-dichlorophenyl)boronic acid.

Melting point (cap): 140-142° C.

Mass spectrometry (ESI) m/z=357.0330 Th ([M+H]⁺)

### EXAMPLE 41

### [1-({[6-Chloro-5-(3,5-dichlorophenyl)pyridin-3-yl]oxy}methyl)cyclopropyl]-methylamine hydrochloride

The compound is obtained in accordance with the procedure of Example 26, with the replacement of (4-fluorophenyl)boronic acid with (3,5-dichlorophenyl)boronic acid.

Melting point (cap): 186-188° C.

Mass spectrometry (ESI) m/z=357.0308 Th ([M+H]⁺)

### EXAMPLE 42

### [1-({[6-Chloro-5-(2,6-dichlorophenyl)pyridin-3-yl]oxy}methyl)cyclopropyl]methylamine hydrochloride

The compound is obtained in accordance with the procedure of Example 26, with the replacement of (4-fluorophenyl)boronic acid with (2,6-dichlorophenyl)boronic acid.

Melting point (cap): 182-186° C.

Mass spectrometry (ESI) m/z=357.0322 Th ([M+H]⁺)

### EXAMPLE 43

### N-[3-(2-Chloro-5-{[1-(methylamino)cyclopropyl]methoxy}pyridin-3-yl)phenyl]-acetamide hydrochloride

The compound is obtained in accordance with the procedure of Example 26, with the replacement of (4-fluorophenyl)boronic acid with ({3-[(methylamino)carbonyl]phenyl})-boronic acid.

Melting point (cap): 220-222° C.

Mass spectrometry (ESI) m/z=346.1324 Th ([M+H]⁺)

### EXAMPLE 44

### [1-({[5-(3-Methylphenyl)pyridin-3-yl]oxy}methyl)cyclopropyl]methylamine dihydrochloride

### Step 1: tert-Butyl[1-({[5-(3-methylphenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (3-methylphenyl)boronic acid.

### Step 2: [1-({[5-(3-Methylphenyl)pyridin-3-yl]oxy}methyl)cyclopropyl]methylamine dihydrochloride

25 ml of 1.5N hydrochloric acid solution in ethanol are added to 0.9 g of the compound obtained in the above Step 1. The mixture is stirred for 20 hours and then 10 ml of 6N hydrochloric acid solution in ethanol are added in order to complete deprotection. After stirring for a further 20 hours, the reaction mixture is made up to 100 ml with ether and the precipitate is filtered off and dried to obtain 0.64 g of the expected product.

Melting point (cap): 215-218° C.

Mass spectrometry (ESI) m/z=269.1655 Th ([M+H]⁺)

### EXAMPLE 45

### [1-({[5-(3-Nitrophenyl)pyridin-3-yl]oxy}methyl)cyclopropyl]methylamine dihydrochloride

### Step 1: tert-Butyl[1-({[5-(3-nitrophenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (3-nitrophenyl)boronic acid.

### Step 2: [1-({[5-(3-Nitrophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 220-225° C.

Mass spectrometry (ESI) m/z=300.1347 Th ([M+H]⁺)

### EXAMPLE 46

### [1-({[5-(3-Chlorophenyl)pyridin-3-yl]oxy}methyl)cyclopropyl]methylamine dihydrochloride

### Step 1: tert-Butyl[1-({[5-(3-chlorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (3-chlorophenyl)boronic acid.

### Step 2: [1-({[5-(3-Chlorophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 230-235° C.

Mass spectrometry (ESI) m/z =289.1074 Th ([M+H]⁺)

### EXAMPLE 47

### [1-({[5-(3-Fluorophenyl)pyridin-3-yl]oxy}methyl)cyclopropyl]methylamine dihydrochloride

### Step 1: tert-Butyl[1-({[5-(3-fluorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (3-fluorophenyl)boronic acid.

### Step 2: [1-({[5-(3-Fluorophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 210-214° C.

Mass spectrometry (ESI) m/z=273.1387 Th ([M+H]⁺)

### EXAMPLE 48

### Methyl{1-[({5-[(4-(methylthio)phenyl]pyridin-3-yl}oxy)methyl]cyclopropyl}amine dihydrochloride

### Step 1: tea-Butyl methyl{4-[({5-[4-(methylthio)phenyl]pyridin-3-yl}oxy)methyl]-cyclopropyl}carbamate

The compound is obtained in accordance with the procedure of Step 1 of Example 1, with the replacement of (3-methoxyphenyl)boronic acid with [4-(methylthio)phenyl]boronic acid.

### Step 2: Methyl{1-[({5-[4-(methylthio)phenyl]pyridin-3-yl}oxy)methyl]cyclopropyl}-amine dihydrochloride

The compound is obtained in accordance with the procedure of Step 2 of Example 44, using the compound obtained in the above Step 1.

Melting point (cap): 232-235° C.

Mass spectrometry (ESI) m/z=301.1367 Th ([M+H]⁺)

### EXAMPLE 49

### [1-({[5-(4-Ethylphenyl)pyridin-3-yl]oxy}methyl)cyclopropyl]methylamine dihydrochloride

### Step 1: tert-Butyl[1-({[5-(4-ethylphenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (4-ethylphenyl)boronic acid.

### Step 2: [1-({[5-(4-Ethylphenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 197-200° C.

Mass spectrometry (ESI) m/z=283.1796 Th ([M+H]⁺)

### EXAMPLE 50

### Methyl[1-({[5-(2-methylphenyl)pyridin-3-yl]oxy}methyl)cyclopropyl]-amine dihydrochloride

### Step 1: tert-Butyl methyl[1-({[5-(2-methylphenyl)pyridin-3-yl]oxy}methyl)cyclopropyl]-carbamate

The compound is obtained in accordance with the procedure of Step 1 of Example 1, with the replacement of (3-methoxyphenyl)boronic acid with (2-methylphenyl)boronic acid.

### Step 2: Methyl[1-({[5-(2-methylphenyl)pyridin-3-yl]oxy}methyl)cyclopropyl]amine dihydrochloride

The compound is obtained in accordance with the procedure of Step 2 of Example 44, using the compound obtained in the above Step 1.

Melting point (cap): 192-196° C.

Mass spectrometry (ESI) m/z=269.1660 Th ([M+H]⁺)

### EXAMPLE 51

### [1-({[5-(2,5-Difluorophenyl)pyridin-3-yl]oxy}methyl)cyclopropyl]methylamine dihydrochloride

### Step 1: tert-Butyl[1-({[5-(2,5-difluorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (2,5-difluorophenyl)boronic acid.

### Step 2: [1-({[5-(2,5-Difluorophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 188-192° C.

Mass spectrometry (ESI) m/z=291 Th ([M+H]⁺)

### EXAMPLE 52

### [1-({[5-(3,5-Dichlorophenyl)pyridin-3-yl]oxy}methyl)cyclopropyl]methylamine dihydrochloride

### Step 1: tert-Butyl[1-({[5-(3,5-dichlorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (3,5-dichlorophenyl)boronic acid.

### Step 2: [1-({[5-(3,5-Dichlorophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 215-220° C.

Mass spectrometry (ESI) m/z=323 Th ([M+H]⁺)

### EXAMPLE 53

### Methyl[1-({[5-(3,4,5-trimethoxyphenyl)pyridin-3-yl]oxy}methyl)cyclopropyl]amine hydrochloride

### Step 1: tert-Butyl methyl[1-({[5-(3,4,5-trimethoxyphenyl)pyridin-3-yl]oxy}methyl)cyclopropyl]carbamate

The compound is obtained in accordance with the procedure of Step 1 of Example 1, with the replacement of (3-methoxyphenyl)boronic acid with (3,4,5-trimethoxyphenyl)boronic acid.

### Step 2: Methyl[1-({[5-(3,4,5-trimethoxyphenyl)pyridin-3-yl]oxy}methyl)cyclopropyl]amine hydrochloride

The compound is obtained in accordance with the procedure of Step 2 of Example 44, using the compound obtained in the above Step 1.

Melting point (cap): 116-120° C.

Mass spectrometry (ESI) m/z=345.1801 Th ([M+H]⁺)

### EXAMPLE 54

### [1-({[5-(2,5-Dichlorophenyl)pyridin-3-yl]oxy}methyl)cyclopropyl]methylamine dihydrochloride

### Step 1: tert-Butyl[1-({[5-(2,5-dichlorophenyl)pyridin-3-yl]oxy}methyl)cyclopropyl]-methylcarbamate

The compound is obtained in accordance with the procedure of Step 1 of Example 1, with the replacement of (3-methoxyphenyl)boronic acid with (2,5-dichlorophenyl)boronic acid.

### Step 2: [1-({[5-(2,5-Dichlorophenyl)pyridin-3-yl]oxy}methyl)cyclopropyl]methylamine dihydrochloride

The compound is obtained in accordance with the procedure of Step 2 of Example 44, using the compound obtained in the above Step 1.

Melting point (cap): 195-200° C.

Mass spectrometry (ESI) m/z=323 Th ([M+H]⁺)

### EXAMPLE 55

### [1-({[5-(2,6-Dichlorophenyl)pyridin-3-yl]oxy}methyl)cyclopropyl]methylamine dihydrochloride

### Step 1: tert-Butyl[1-({[5-(2,6-dichlorophenyl)pyridin-3-yl]oxy}methyl)cyclopropyl]-methylcarbamate

The compound is obtained in accordance with the procedure of Step 1 of Example 1; with the replacement of (3-methoxyphenyl)boronic acid with (2,6-dichlorophenyl)boronic acid.

### Step 2: [1-({[5-(2,6-Dichlorophenyl)pyridin-3-yl]oxy}methyl)cyclopropyl]methylamine dihydrochloride

The compound is obtained in accordance with the procedure of Step 2 of Example 44, using the compound obtained in the above Step 1.

Mass spectrometry (ESI) m/z=323.0699 Th ([M+H]⁺)

Example 56

### {1-[({5-[3,5-bis(Trifluoromethyl)phenyl]pyridin-3-yl}oxy)methyl]cyclopropyl}-methylamine hydrochloride

### Step 1: tert-Butyl{1-[({5-[3,5-bis(trifluoromethyl)phenyl]pyridin-3-yl}oxy)methyl]-cyclopropyl}methylcarbamate

The compound is obtained in accordance with the procedure of Step 1 of Example 1, with the replacement of (3-methoxyphenyl)boronic acid with [3,5-bis(trifluoromethyl)phenyl]-boronic acid.

### Step 2: {1[({5-[3,5-bis(Trifluoromethyl)phenyl]pyridin-3-yl}oxy)methyl]cyclopropyl}-methylamine hydrochloride

The compound is obtained in accordance with the procedure of Step 2 of Example 44, using the compound obtained in the above Step 1.

Melting point (cap): 132-136° C.

Mass spectrometry (ESI) m/z=391.1249 Th ([M+H]⁺)

### EXAMPLE 57

### Methyl[1-({[5-(2,3,4-trimethoxyphenyl)pyridin-3-yl]oxy}methyl)cyclopropyl]amine dihydrochloride

### Step 1: tert-Butyl methyl[1-({[5-(2,3,4-trimethoxyphenyl)pyridin-3-yl]oxy}methyl)-cyclopropyl]carbamate

The compound is obtained in accordance with the procedure of Step 1 of Example 1, with the replacement of (3-methoxyphenyl)boronic acid with (2,3,4-trimethoxyphenyl)boronic acid.

### Step 2: Methyl[1-({[5-(2,3,4-trimethoxyphenyl)pyridin-3-yl]oxy}methylcyclopropyl]-amine dihydrochloride

The compound is obtained in accordance with the procedure of Step 2 of Example 44, using the compound obtained in the above Step 1.

Mass spectrometry (ESI) m/z=345.1783 Th ([M+H]⁺)

### EXAMPLE 58

### N-[3-(5-{[1-(Methylamino)cyclopropyl]methoxy}pyridin-3-yl)phenyl]acetamide dihydrochloride

### Step 1: tert-Butyl{1-[({5-[3-(acetylamino)phenyl]pyridin-3-yl}oxy)methyl]cyclopropyl}-methylcarbamate

The compound is obtained in accordance with the procedure of Step 1 of Example 1, with the replacement of (3-methoxyphenyl)boronic acid with [3-(acetylamino)phenyl]boronic acid.

### Step 2: N-[3-(5-{[1-(Methylamino)cyclopropyl]methoxy}pyridin-3-yl)phenyl]acetamide dihydrochloride

1.05 g of the product obtained in the above Step 1 are dissolved in 5 ml of ethanol and 15 ml of 5N hydrochloric acid solution in ethanol are added. After stirring for 20 hours, the reaction mixture is concentrated to three quarters and diluted with ether. The precipitate is taken up in saturated sodium carbonate solution and extracted with ethyl acetate. The organic phase is dried over sodium sulphate and then evaporated. The mixture obtained is chromatographed on 40 g of silica gel to isolate 0.13 g of the base of the expected product. The base is taken up in ethanol and, after addition of hydrochloric acid solution in ether and filtration, the solid collected is recovered and lyophilised to obtain 0.14 g of the expected product.

Mass spectrometry (ESI) m/z=312.1704 Th ([M+H]⁺)

### EXAMPLE 59

### [1-({[5-(3-Aminophenyl)pyridin-3-yl]oxy}methyl)cyclopropyl]methylamine trihydrochloride

### Step 1: tert Butyl[1-({[5-(3-aminophenyl)pyridin-3-yl]oxy}methyl)cyclopropyl]methylcarbamate

The compound is obtained in accordance with the procedure of Step 1 of Example 1, with the replacement of (3-methoxyphenyl)boronic acid with (3-aminophenyl)boronic acid.

### Step 2: [1-({[5-(3-Aminophenyl)pyridin-3-yl]oxy}methyl)cyclopropyl]methylamine trihydrochloride

The compound is obtained in accordance with the procedure of Step 2 of Example 44, using the compound obtained in the above Step 1.

Melting point (cap): 185-190° C.

Mass spectrometry (ESI) m/z=270.1604 Th ([M+H]⁺)

### EXAMPLE 60

### [1-({[5-(3-Aminophenyl)-6-fluoropyridin-3-yl]oxy}methyl)cyclopropyl]methylamine dihydrochloride

### Step 1: tert-Butyl[1-({[5-(3-aminophenyl)-6-fluoropyridin-3-yl]oxy}methyl)cyclopropyl]methylcarbamate

The compound is obtained in accordance with the procedure of Step 1 of Example 1, using the compound of Preparation 4 instead of the compound of Preparation 1 and with the replacement of (3-methoxyphenyl)boronic acid with (3-aminophenyl)boronic acid.

### Step 2: [1-({[5-(3-Aminophenyl)-6-fluoropyridin-3-yl]oxy}methyl)cyclopropyl]-methylamine dihydrochloride

75 ml of dioxane and then 10 ml of 4N hydrochloric acid solution in dioxane are added to 1.03 g of the product obtained in the above Step 1. 50 ml of ethanol are added to effect complete homogenisation of the reaction mixture. After stirring for 20 hours, the solvents are evaporated off, and the residue is taken up in sodium carbonate solution and extracted with dichloromethane. After drying over sodium sulphate, the organic phase is concentrated and the residue obtained is chromatographed on silica gel (dichloro-methane/methanol: 97/3) to obtain 0.53 g of the base of the expected product. After the addition of hydrochloric acid solution in ether and filtration, 0.5 g of the expected product is obtained.

Melting point (cap): 158-161° C.

Mass spectrometry (ESI) m/z=288.1488 Th ([M+H]⁺)

### EXAMPLE 61

### [1-({[5-(3-Aminophenyl)-6-methylpyridin-3-yl]oxy}methyl)cyclopropyl]methylamine trihydrochloride

### Step 1: tert-Butyl[1-({[5-(3-aminophenyl)-6-methylpyridin-3-yl]oxy}methyl)cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (3-aminophenyl)boronic acid.

### Step 2: [1-({[5-(3-Aminophenyl)-6-methylpyridin-3-yl]oxy}methyl)cyclopropyl]-methylamine trihydrochloride

40 ml of dioxane and then 10 ml of 4N hydrochloric acid solution in dioxane are added to 1.05 g of the product obtained in the above Step 1. 40 ml of ethanol are added to effect complete homogenisation of the reaction mixture. After stirring for 20 hours, the solvents are evaporated off and the residue is taken up in a minimum of ethanol. After dilution with ether and filtration, 0.71 g of the expected product is obtained.

Melting point (cap): 222-228° C.

Mass spectrometry (ESI) m/z=284.1783 Th ([M+H]⁺)

### EXAMPLE 62

Ethyl 4-(2-chloro-5-{[1-(methylamino)cyclopropyl]methoxy}pyridin-3-yl)benzoate dihydrochloride

### Step 1: Ethyl 4-[5-({1-[(tert-butoxycarbonyl)(methyl)amino]cyclopropyl}methoxy)-2-chloropyridin-3-yl]benzoate

The compound is obtained in accordance with the procedure of Step 1 of Example 1, using the compound of Preparation 2 instead of the compound of Preparation 1 and with the replacement of (3-methoxyphenyl)boronic acid with (4-ethoxycarbonylphenyl)boronic acid.

### Step 2: Ethyl 4-(2-chloro-5-{[1-(methylamino)cyclopropyl]methoxy}pyridin-3-yl)-benzoate dihydrochloride

5 ml of 4N hydrochloric acid solution in dioxane are added to a solution of 0.5 g of the product obtained in the above Step 1 in 5 ml of dioxane and 5 ml of ethanol. After stirring for 20 hours, deprotection is complete and the reaction mixture is concentrated. The residue obtained is chromatographed on an RP 18 column 12-25μ (water/trifluoroacetic acid: 1000/2.5 to water/acetonitrile/trifluoroacetic acid: 450/550/2.5). The chromatography fractions are combined and then the acetonitrile is evaporated off. The residual aqueous solution is neutralised and then saturated with solid sodium hydrogen carbonate and subsequently extracted with ethyl acetate. After drying over sodium sulphate and concentrating the organic phase, the base obtained is dissolved in ethanol and 4N hydrochloric acid solution in dioxane is added. After concentration and crystallisation there is obtained, after washing with ether and drying, 0.25 g of the expected product.

Melting point (cap): 146-148° C.

Mass spectrometry (ESI) m/z=361.1357 Th ([M+H]⁺)

### EXAMPLE 63

### Ethyl 3-(2-chloro-5-({[1-(methylamino)cyclopropyl]methoxy}pyridin-3-yl)benzoate dihydrochloride

### Step 1: Ethyl 3-[5-({1-[(tert-butoxycarbonyl)(methyl)amino]cyclopropyl}methoxy)-2-chloropyridin-3-yl]benzoate

The compound is obtained in accordance with the procedure of Step 1 of Example 1, using the compound of Preparation 2 instead of the compound of Preparation 1 and with the replacement of (3-methoxyphenyl)boronic acid with (3-ethoxycarbonylphenyl)boronic acid.

### Step 2: Ethyl 3-(2-chloro-5-{[1-(methylamino)cyclopropyl]methoxy}pyridin-3-yl)-benzoate dihydrochloride

The compound is obtained in accordance with the procedure of Step 2 of Example 62, using the compound obtained in the above Step 1. The final product is taken up in water and then lyophilised.

Mass spectrometry (ESI) m/z=361.1328 Th ([M+H]⁺)

### EXAMPLE 64

### [1-({[6-Chloro-5-(4-methoxyphenyl}pyridin-3-yl]oxy)methyl)cyclopropyl]amine dihydrochloride

### Step 1: tert-Butyl[1-({[6-chloro-5-(4-methoxyphenyl)pyridin-3-yl]oxy}methyl)cyclopropyl]carbamate

The compound is obtained in accordance with the procedure of Step 1 of Example 1, using the compound of Preparation 2 instead of the compound of Preparation 1 and with the replacement of (3-methoxyphenyl)boronic acid with (4-methoxyphenyl)boronic acid.

Melting point (cap): 131° C.

### Step 2: [1-({[6-Chloro-5-(4-methoxyphenyl)pyridin-3-yl]oxy}methyl)cyclopropyl]amine dihydrochloride

2 ml of 4N hydrochloric acid solution in dioxane are added to a solution of 1.64 g of the product obtained in the above Step 1 in 40 ml of methanol. After stirring for 20 hours, the solvents are evaporated off and the residue is triturated in ether and then filtered. The solid is taken up in 20 ml of 1.5N hydrochloric acid solution in methanol. Heating is carried out until dissolution occurs, and then the reaction mixture, allowed to return to ambient temperature, crystallises. After filtration and drying, 0.95 g of the expected product is obtained.

Melting point (cap): 207-211° C.

Mass spectrometry (ESI) m/z=305.1046 Th ([M+H]⁺)

### EXAMPLE 65

### [1-({[6-Chloro-5-(4-methoxyphenyl)pyridin-3-yl]oxy}methyl)cyclopropyl]-dimethylamine hydrochloride

A solution of 1.28 g of the compound of Step 1 of Example 11 in 12.5 ml of formic acid is stirred for 2 hours at ambient temperature. 12.5 ml of 40% formaldehyde solution in water are added and then the reaction mixture is heated for 2 hours at 70° C. 2.5 ml of formic acid and an additional 2.5 ml of the formaldehyde solution are added to the reaction mixture, and heating at 70° C. is continued for 2 hours. The reaction mixture is concentrated, and then 15 ml of saturated aqueous potassium carbonate solution are added and extraction is carried out with dichloromethane. The dichloromethane is dried over sodium sulphate and evaporated. The residue obtained is chromatographed on silica gel (dichloromethane/methanol: 97/3) to isolate 0.63 g of base. 1.1 ml of 4N hydrochloric acid solution in dioxane are added to a solution in 5 ml of ethanol of the above isolated base. The reaction mixture is diluted with 100 ml of ether and stirred for 30 min. After filtration and drying, 0.57 g of the expected product is obtained.

Melting point (cap): 208-210° C.

Mass spectrometry (ESI) m/z=333.1377 Th ([M+H]⁺)

### EXAMPLE 66

### [1-({[6-Chloro-5-(chloromethyl)pyridin-3-yl]oxy}methyl)cyclopropyl]methylamine hydrochloride

### Step 1: tert-Butyl 1-({[6-chloro-5-(chloromethyl)pyridin-3-yl]oxy}methyl)cyclopropyl]methylcarbamate

1.65 g of triphenylphosphine are added to a solution of 2 g of the compound obtained in Step 1 of Example 14 in 10 ml of carbon tetrachloride. The mixture is heated at reflux until starting material is no longer present in thin-layer chromatography and is then cooled to ambient temperature and filtered. The filtrate is concentrated and the residue obtained is chromatographed on silica gel (dichloromethane/tetrahydrofuran: 97/3) to obtain 1.8 g of the expected product.

### Step 2: [1-({[6-chloro-5-(Chloromethyl)pyridin-3-yl]oxy}methyl)cyclopropyl]-methylamine hydrochloride

The compound is obtained in accordance with the procedure of Step 2 of Example 2, using the compound obtained in the above Step 1.

Melting point (cap): 136-140° C.

### EXAMPLE 67

### 4-(2-Chloro-5-{[1-(methylamino)cyclopropyl]methoxy}pyridin-3-yl)benzamide hydrochloride

1.2 g of potassium carbonate are added to a solution of 2.2 g of the compound obtained in Step 1 of Example 18 in 30 ml of dimethyl sulphoxide. The reaction mixture is cooled to from 0 to 5° C. with a mixture of water and ice and then 3.6 ml of aqueous 30% hydrogen peroxide solution are added dropwise. After stirring for 45 min., the reaction mixture is diluted with water and then filtered. The solid collected is washed with water and taken up in ethyl acetate. The ethyl acetate solution is dried over sodium sulphate and then concentrated. 2.1 g of crude intermediate, tert-butyl [1-({[5-(aminocarbonyl)-6-chloropyridin-3-yl]oxy}methyl)cyclopropyl]methylcarbamate, are taken up in 10 ml of ethanol and then 15 ml of 4N hydrochloric acid solution in dioxane are added. After stirring for 20 hours, the reaction mixture is diluted with ether and then filtered. The solid collected is chromatographed on an RP18 column, 12-25μ (water/trifluoroacetic acid: 1000/2.5 to water/acetonitrile/trifluoroacetic acid: 675/325/2.5). The chromatography fractions are combined and then the acetonitrile is evaporated off. The residual aqueous solution is neutralised and then saturated with solid sodium hydrogen carbonate and subsequently extracted with dichloromethane. After drying over sodium sulphate and concentrating the organic phase, the base obtained is dissolved in 25 ml of ethanol and 1 ml of 4N hydrochloric acid solution in dioxane is added. After concentration and crystallisation in ether, the crystals are filtered off and dried to obtain 1.1 g of the expected product.

Melting point (cap): 130° C.

Mass spectrometry (ESI) m/z=332.1184 Th ([M+H]⁺)

### EXAMPLE 68

### 3-(5-{[1-(Methylamino)cyclopropyl]methoxy}pyridin-3-yl)benzoic acid dihydrochloride

### Step 1: 3-[5-({1-[(tert-Butoxycarbonyl)(methyl)amino]cyclopropyl}methoxy)pyridin-3-yl]benzoic acid

25 ml of aqueous 0.4M sodium carbonate solution and 0.58 g of (3-carboxyphenyl)boronic acid are added to a solution of 1 g of the compound of Preparation 1 in 25 ml of acetonitrile. After stirring for 45 min. under argon, 0.15 g of tetrakis(triphenylphosphine)-palladium is added and then the mixture is heated for 5 hours 30 minutes at 80° C. The reaction mixture is filtered hot, and the pH of the cooled filtrate is adjusted to 5.5, using a pH meter, by adding aqueous 1N hydrochloric acid solution. The reaction mixture is extracted with ethyl acetate. The organic phase is dried over sodium sulphate and then concentrated. Chromatography on silica gel (dichloromethane/methanol: 97/3) allows 0.9 g of the expected product to be isolated.

### Step 2: 3-(5-{[1-(Methylamino)cyclopropyl]methoxyl}pyridin-3-yl)benzoic acid dihydrochloride

5 ml of 4N hydrochloric acid solution in dioxane are added to a solution of 0.9 g of the compound obtained in the above Step 1 in 10 ml of dioxane. The mixture is stirred for 20 hours, diluted with ether and then filtered. The solid collected is taken up in 25 ml of water to obtain, after lyophilisation, 0.595 g of the expected product.

Mass spectrometry (ESI) m/z=299.1374 Th ([M+H]⁺)

### EXAMPLE 69

### 4-(5-{[1-(Methylamino)cyclopropyl]methoxy}pyridin-4-yl)benzoic acid dihydrochloride

### Step 1: 4-[5-({1-[(tert-Butoxycarbonyl)(methyl)amino]cyclopropyl}methoxy)pyridin-3-yl]benzoic acid

The compound is obtained in accordance with the procedure of Step 1 of Example 68, with the replacement of (3-carboxyphenyl)boronic acid with (4-carboxyphenyl)boronic acid.

### Step 2: 4-(5-{[1-(Methylamino)cyclopropyl]methoxy}pyridin-4-yl)benzoic acid dihydrochloride

The compound is obtained in accordance with the procedure of Step 2 of Example 68, using the compound obtained in the above Step 1.

Mass spectrometry (ESI) m/z=299.1372 Th ([M+H]⁺)

### EXAMPLE 70

### 3-(2-Chloro-5-{[1-(methylamino)cyclopropyl]methoxy}pyridin-3-yl)benzoic acid hydrochloride

### Step 1: 3-[5-({1-[(tert-Butoxycarbonyl)(methyl)amino]cyclopropyl}methoxy)-2-chloropyridin-3-yl]benzoic acid

The compound is obtained in accordance with the procedure of Step 1 of Example 68, using the compound of Preparation 2 instead of the compound of Preparation 1.

### Step 2: 3-(2-Chloro-5-{[1-(methylamino)cyclopropyl]methoxyl}pyridin-3-yl)benzoic acid hydrochloride

The compound is obtained in accordance with the procedure of Step 2 of Example 68, using the compound obtained in the above Step 1.

Mass spectrometry (ESI) =333.1004 Th ([M+H]⁺)

### EXAMPLE 71

### 4-(2-Chloro-5-{[1-(methylamino)cyclopropyl]methoxy}pyridin-3-yl)benzoic acid hydrochloride

### Step 1: 4-[5-({1-[(tert-Butoxycarbonyl)(methyl)amino]cyclopropyl}methoxy)-2-chloropyridin-3-yl]benzoic acid

The compound is obtained in accordance with the procedure of Step 1 of Example 68, using the compound of Preparation 2 instead of the compound of Preparation 1 and with the replacement of (3-carboxyphenyl)boronic acid with (4-carboxyphenyl)boronic acid.

### Step 2: 4-(2-Chloro-5-{[1-(methylamino)cyclopropyl]methoxy}pyridin-3-yl)benzoic acid hydrochloride

The compound is obtained in accordance with the procedure of Step 2 of Example 68, using the compound obtained in the above Step 1.

Mass spectrometry (ESI) m/z=333.1003 Th ([M+H]⁺)

### EXAMPLE 72

### 3-(2-Fluoro-5-{[1-(methylamino)cyclopropyl]methoxy}pyridin-3-yl)benzoic acid dihydrochloride

### Step 1: 3-[5-({1-[(tert-Butoxycarbonyl)(methyl)amino]cyclopropyl}methoxy)-2-fluoropyridin-3-yl]benzoic acid

The compound is obtained in accordance with the procedure of Step 1 of Example 68, using the compound of Preparation 4 instead of the compound of Preparation 1.

### Step 2: 3-(2-Fluoro-5-{[1-(methylamino)cyclopropyl]methoxy}pyridin-3-yl)benzoic acid dihydrochloride

The compound is obtained in accordance with the procedure of Step 2 of Example 68, using the compound obtained in the above Step 1.

Mass spectrometry (ESI) m/z=317.1315 Th ([M+H]⁺)

### EXAMPLE 73

### 4-(2-Fluoro-5-{[1-(methylamino)cyclopropyl]methoxy}pyridin-3-yl)benzoic acid dihydrochloride

### Step 1: 4-[5-{1-[(tert-Butoxycarbonyl)(methyl)amino]cyclopropyl}methoxy)-2-fluoro-pyridin-3-yl]benzoic acid

The compound is obtained in accordance with the procedure of Step 1 of Example 68, using the compound of Preparation 4 instead of the compound of Preparation 1 and with the replacement of (3-carboxyphenyl)boronic acid with (4-carboxyphenyl)boronic acid.

### Step 2: 4-(2-Fluoro-5-{[1-(methylamino)cyclopropyl]methoxy}pyridin-3-yl)benzoic acid dihydrochloride

The compound is obtained in accordance with the procedure of Step 2 of Example 1, using the compound obtained in the above Step 1.

Melting point (cap): 211-215° C.

Mass spectrometry (ESI) m/z=317.1324 Th ([M+H]⁺)

### EXAMPLE 74

### 3-(2-Methyl-5-({[1-(methylamino)cyclopropyl]methoxy}pyridin-3-yl)benzoic acid dihydrochloride

### Step 1: 3-[5-({1-[(tert-Butoxycarbonyl)(methyl)amino]cyclopropyl}methoxy)-2-methylpyridin-3-yl]benzoic acid

The compound is obtained in accordance with the procedure of Step 1 of Example 68, using the compound of Preparation 3 instead of the compound of Preparation 1.

### Step 2: 3-(2-Methyl-5-{[1-(methylamino)cyclopropyl]methoxy}pyridin-3-yl)benzoic acid dihydrochloride

The compound is obtained in accordance with the procedure of Step 2 of Example 1, using the compound obtained in the above Step 1.

Melting point (cap): 210-215° C.

Mass spectrometry (ESI) m/z=313.1515 Th ([M+H]⁺)

### EXAMPLE 75

### 4-(2-Methyl-5-({[1-(methylamino)cyclopropyl]methoxy}pyridin-3-yl)benzoic acid dihydrochloride

### Step 1: 4-[5-({1-[(tert-Butoxycarbonyl)(methyl)amino]cyclopropyl}methoxy)-2-methylpyridin-3-yl]benzoic acid

The compound is obtained in accordance with the procedure of Step 1 of Example 68, using the compound of Preparation 3 instead of the compound of Preparation 1 and with the replacement of (3-carboxyphenyl)boronic acid with (4-carboxyphenyl)boronic acid.

### Step 2: 4-(2-Methyl-5-{[1-(methylamino)cyclopropyl]methoxy}pyridin-3-yl)benzoic acid dihydrochloride

The compound is obtained in accordance with the procedure of Step 2 of Example 1, using the compound obtained in the above Step 1.

Melting point (cap): 228-235° C.

Mass spectrometry (ESI) m/z=313.1580 Th ([M+H]⁺)

### EXAMPLE 76

### (1-{[(2-Chloro-3,3′-bipyridin-5-yl)oxy]methyl}cyclopropyl)methylamine dihydrochloride

### Step 1: tert-Butyl(1-{[1(2-chloro-3,3′-bipyridin-5-yl)oxy]methyl}cyclopropyl)-methylcarbamate

0.87 g of tetrakis(triphenylphosphine)palladium are added under nitrogen to a solution of 2.91 g of the compound of Preparation 2 in 45 ml of toluene. The mixture is stirred for 20 minutes and then a solution of 3 g of 3-(1,1,1-tributylstannyl)pyridine in 6 ml of toluene is added and the reaction mixture is heated at reflux for 20 hours. A second fraction of 0.87 g of Pd(PPh₃)₄ is added and refluxing is continued for 24 hours. After cooling, the reaction mixture is diluted with 120 ml of toluene and then washed with aqueous 50% potassium carbonate solution. The organic phase is dried over sodium sulphate and concentrated. Chromatography on silica gel (dichloromethane/butanone: 97/3 to dichloromethane/butanone: 80/20) allows 1.76 g of the expected product to be obtained.

### Step 2: (1-{[(2-Chloro-3,3′-bipyridin-5-yl)oxy]methyl}cyclopropyl)methylamine dihydrochloride

24.6 ml of 4N hydrochloric acid solution in dioxane are added to a solution of 2.46 g of the compound obtained in the above Step 1 in 24.6 ml of methanol. After stirring for 20 hours, the solvents are evaporated off and the residue is taken up in aqueous 50% potassium carbonate solution and extracted with dichloromethane. The organic phase is dried over sodium sulphate and concentrated. The residue is chromatographed on silica gel (dichloromethane/methanol: 97/3) to isolate 1.05 g of base. The base is taken up in 120 ml of ethanol and hydrochloric acid solution in ethanol is added until an acid pH is obtained. The reaction mixture is concentrated and then triturated in ether to obtain, after crystallisation, filtration and drying, 1.15 g of the expected product.

Melting point (cap): 210-215° C.

Mass spectrometry (ESI) m/z=290.1037 Th ([M+H]⁺)

### EXAMPLE 77

### (1-{[(2-Chloro-3,4′-bipyridin-5-yl)oxy]methyl}cyclopropyl)methylamine dihydrochloride

### Step 1: tert-Butyl(1-{[(2-chloro-3,4′-bipyridin-5-yl)oxy]methyl}cyclopropyl)-methylcarbamate

The compound is obtained in accordance with the procedure of Step 1 of Example 76, with the replacement of 3-(1,1,1-tributylstannyl)pyridine with 4-(1,1,1-tributylstannyl)pyridine.

### Step 2: (1-{[(2-Chloro-3,4′-bipyridin-5-yl)oxy]methyl}cyclopropyl)methylamine dihydrochloride

The compound is obtained in accordance with the procedure of Step 2 of Example 76, using the compound obtained in the above Step 1.

Melting point (cap): 125-130° C.

Mass spectrometry (ESI) m/z=290.1035 Th ([M+H]⁺)

### EXAMPLE 78

### (1-{[(2-Fluoro-3,3′-bipyridin-5-yl)oxy]methyl}cyclopropyl)methylamine dihydrochloride

### Step 1: tert-Butyl(1-{[(2-fluoro-3,3′-bipyridin-5-yl)oxy]methyl}cyclopropyl)-methylcarbamate

0.76 g of tetrakis(triphenylphosphine)palladium are added under nitrogen to a solution of 2.5 g of the compound of Preparation 4 in 40 ml of toluene. The mixture is stirred for 20 minutes and then a solution of 2.6 g of 3-(1,1,1-tributylstannyl)pyridine in 5 ml of toluene is added and the reaction mixture is heated at reflux for 20 hours. After cooling, the reaction mixture is diluted with toluene and then washed with aqueous 50% potassium carbonate solution. The organic phase is dried over sodium sulphate and concentrated. Chromatography on silica gel (dichloromethane/butanone: 97/3 to dichloromethane/-butanone: 90/10) allows 1.66 g of the expected product to be obtained.

### Step 2: (1-{[(2-Fluoro-3,3′-bipyridin-5-yl)oxy]methyl}cyclopropyl)methylamine dihydrochloride

16 ml of 4N hydrochloric acid solution in dioxane are added to a solution of 1.6 g of the compound obtained in the above Step 1 in 100 ml of ethanol. After stirring for 20 hours, the solvents are evaporated off, the residue is dissolved in a minimum of ethanol and ether is added. Stirring is carried out for 20 hours, the solvents are decanted off and ether is added again to the solid residue to obtain, after crystallisation, filtration and drying, 0.7 g of the expected compound.

Mass spectrometry (ESI) m/z=274.1365 Th ([M+H]⁺)

### EXAMPLE 79

### (1-{[(2-Fluoro-3,4′-bipyridin-5-yl)oxy]methyl}cyclopropyl)methylamine dihydrochloride

### Step 1: tert-Butyl(1-{[(2-fluoro-3,4′-bipyridin-5-yl)oxy]methyl}cyclopropyl)-methylcarbamate

The compound is obtained in accordance with the procedure of Step 1 of Example 78, with the replacement of 3-(1,1,1-tributylstannyl)pyridine with 4-(1,1,1-tributylstannyl)pyridine.

### Step 2: (1-{[(2-Fluoro-3,4′-bipyridin-5-yl)oxy]methyl}cyclopropyl)methylamine dihydrochloride

24 ml of 4N hydrochloric acid solution in dioxane are added to a solution of 2.4 g of the compound obtained in the above Step 1 in 120 ml of ethanol After stirring for 20 hours, the solvents are evaporated off and the residue is taken up in aqueous 50% potassium carbonate solution and extracted with dichloromethane. The organic phase is dried over sodium sulphate and concentrated. The residue is chromatographed on silica gel (dichloromethane/methanol: 97/3). The base is dissolved in a minimum of ethanol and hydrochloric acid solution in ether is added until an acid pH is obtained. The reaction mixture is concentrated and then triturated in ether to obtain, after crystallisation, filtration and drying, 1 g of the expected product.

Mass spectrometry (ESI) m/z=274.1344 Th ([M+H]⁺)

### EXAMPLE 80

### Methyl(1-{[(2-methyl-3,3′-bipyridin-5-yl)oxy]methyl}cyclopropyl)amine trihydrochloride

### Step 1: tert-Butyl methyl(1-{[(2-methyl-3,3′-bipyridin-5-yl)oxy]methyl}cyclopropyl)-carbamate

The compound is obtained in accordance with the procedure of Step 1 of Example 78, using the compound of Preparation 3 instead of the compound of Preparation 4.

### Step 2: Methyl(1-{[(2-methyl-3,3′-bipyridin-5-yl)oxy]methyl}cyclopropyl)amine trihydrochloride

The compound is obtained in accordance with the procedure of Step 2 of Example 78, using the compound obtained in the above Step 1.

Mass spectrometry (ESI) m/z=270.1619 Th ([M+H]⁺)

### EXAMPLE 81

### Methyl(1-{[(2-methyl-3,4′-bipyridin-5-yl)oxy]methyl}cyclopropyl)amine trihydrochloride

### Step 1: tert-Butyl methyl(1-{[(2-methyl-3,4′-bipyridin-5-yl)oxy]methyl}cyclopropyl)-carbamate

The compound is obtained in accordance with the procedure of Step 1 of Example 78, using the compound of Preparation 3 instead of the compound of Preparation 4 and with the replacement of 3-(1,1,1-tributylstannyl)pyridine with 4-(1,1,1-tributylstannyl)pyridine.

### Step 2: Methyl(1-{[(2-methyl-3,4′-bipyridin-5-yl)oxy]methyl}cyclopropyl)amine trihydrochloride

The compound is obtained in accordance with the procedure of Step 2 of Example 78, using the compound obtained in the above Step 1.

Melting point (cap): 130-138° C.

Mass spectrometry (ESI) m/z=270.1609 Th ([M+H]⁺)

### EXAMPLE 82

### [1-({[5-(4-Aminophenyl)-6-chloropyridin-3-yl]oxy}methyl)cyclopropyl]methylamine dihydrochloride

### Step 1: tert-Butyl [1-({[5-(4-aminophenyl)-6-chloropyridin-3-yl]oxy}methyl)cyclopropyl]methylcarbamate

0.67 g of tetrakis(triphenylphosphine)palladium are added under nitrogen to a solution of 1 g of the compound of Preparation 2 and 4-(4,4,5,5-tetramethyl-1,3,2-dioxaborolan-2-yl)aniline in 30 ml of tetrahydrofuran. The mixture is stirred for 20 minutes and then a solution of 1.65 g of sodium carbonate in 10 ml of water is added and the reaction mixture is heated for 20 hours at 60° C. The solvents are evaporated off and then the residue is taken up in sodium carbonate solution and extracted with dichloromethane. The organic phase is dried over sodium sulphate and concentrated. Chromatography on silica gel (dichloromethane/ethyl acetate: 98/2 to 90/10) allows 0.87 g of the expected product to be obtained.

### Step 2: [1-({[5-(4-Aminophenyl)-6-chloropyridin-3-yl]oxy}methyl)cyclopropyl]-methylamine dihydrochloride

The compound is obtained in accordance with the procedure of Step 2 of Example 2, using the compound obtained in the above Step 1.

Melting point (cap): 240-243° C.

Mass spectrometry (ESI) m/z=304 Th ([M+H]⁺)

### EXAMPLE 83

### [1-({[5-(4-Aminophenyl)-6-fluoropyridin-3-yl]oxy}methyl)cyclopropyl]methylamine dihydrochloride

### Step 1: tert-Butyl[1-({[5-(4-aminophenyl)-6-fluoropyridin-3-yl]oxy}methyl)cyclopropyl]methylcarbamate

The compound is obtained in accordance with the procedure of Step 1 of Example 82, using the compound of Preparation 4 instead of the compound of Preparation 2.

### Step 2: [1-({[5-(4-Aminophenyl)-6-fluoropyridin-3-yl]oxy}methyl)cyclopropyl]-methylamine dihydrochloride

The compound is obtained in accordance with the procedure of Step 2 of Example 22, using the compound obtained in the above Step 1.

Melting point (cap): 225-230° C.

Mass spectrometry (ESI) m/z=288.1521 Th ([M+H]⁺)

### EXAMPLE 84

### 4-{5-[(1-Aminocyclopropyl)methoxy]-2-chloropyridin-3-yl}phenol dihydrochloride

### Step 1: tert-Butyl[1-({[6-chloro-5-(4-hydroxyphenyl)pyridin-3-yl]oxy}methyl)cyclopropyl]carbamate

The compound is obtained in accordance with the procedure of Step 1 of Example 82, using the compound of Preparation 5 instead of the compound of Preparation 2 and with the replacement of 4-(4,4,5,5-tetramethyl-1,3,2-dioxaborolan-2-yl)aniline with 4-(4,4,5,5-tetramethyl-1,3,2-dioxaborolan-2-yl)phenol.

### Step 2: 4-{5-[(1-Aminocyclopropyl)methoxy]-2-chloropyridin-3-yl}phenol dihydrochloride

The compound is obtained in accordance with the procedure of Step 2 of Example 1, using the compound obtained in the above Step 1.

Melting point (cap): decomposition >200° C.

Mass spectrometry (ESI) m/z=291.0894 Th ([M+H]⁺)

### EXAMPLE 85

### 4-(2-Chloro-5-{[1-(methylamino)cyclopropyl]methoxy}pyridin-3-yl)phenol dihydrochloride

### Step 1: tert-Butyl[1-({[6-chloro-5-(4-hydroxyphenyl)pyridin-3-yl]oxy}methyl)cyclopropyl]methylcarbamate

The compound is obtained in accordance with the procedure of Step 1 of Example 82, with the replacement of 4-(4,4,5,5-tetramethyl-1,3,2-dioxaborolan-2-yl)aniline with 4-(4,4,5,5-tetramethyl-1,3,2-dioxaborolan-2-yl)phenol.

### Step 2: 4-(2-Chloro-5-{[1-(methylamino)cyclopropyl]methoxy}pyridin-3-yl)phenol dihydrochloride

The compound is obtained in accordance with the procedure of Step 2 of Example 1, using the compound obtained in the above Step 1.

Melting point (cap): 216-220° C.

Mass spectrometry (ESI) m/z=305.1062 Th ([M+H]⁺)

### EXAMPLE 86

### 2-Chloro-N-[4-(2-chloro-5-{[1-(methylamino)cyclopropyl]methoxy}pyridin-3-yl)phenyl]acetamide hydrochloride

### Step 1: tert-Butyl(1-{[(6-chloro-5-{4-[(chloroacetyl)amino]phenyl}pyridin-3-yl)oxy]methyl}cyclopropyl)methylcarbamate

0.44 g of chloroacetyl chloride are added at 10° C. to a solution of 1.5 g of the compound obtained in Step 1 of Example 82 and of 0.54 ml of triethylamine in 20 ml of tetrahydrofuran. The reaction mixture is stirred for 20 hours at ambient temperature and then concentrated. The residue is taken up in a mixture of ether and water and then concentrated. The organic phase is decanted off and then dried over sodium sulphate and concentrated to obtain 1.7 g of the expected product.

### Step 2: 2-Chloro-N-[4-(2-chloro-5-{[1-(methylamino)cyclopropyl]methoxy}pyridin-3-yl)phenyl]acetamide hydrochloride

The compound is obtained in accordance with the procedure of Step 2 of Example 1, using the compound obtained in the above Step 1.

Melting point (cap): 140-144° C.

Mass spectrometry (ESI) m/z=380.0928 Th ([M+H]⁺)

### EXAMPLE 87

### [1-({[6-Chloro-5-(4-isothiocyanatophenyl)pyridin-3-yl]oxy}methyl)cyclopropyl]-methylamine hydrochloride

### Step 1: tert-Butyl[1-({[6-chloro-5-(4-isothiocyanatophenyl)pyridin-3-yl]oxy}methyl)-cyclopropyl]methylcarbamate

A solution of 1.05 g of sodium hydrogen carbonate in 20 ml of water is added to a solution of 1.01 g of the compound obtained in Step 1 of Example 82 in 20 ml of tetrahydrofuran. 1.38 g of thiophosgene are subsequently added dropwise and then the orange reaction mixture is stirred for one hour at ambient temperature. 30 ml of saturated aqueous sodium hydrogen carbonate solution are added and the reaction mixture is extracted with dichloromethane. The organic phase is dried over sodium sulphate and concentrated. Chromatography on silica gel (dichloromethane/tetrahydrofuran: 98/2) allows 1 g of the expected product to be obtained.

### Step 2: [1-({[6-Chloro-5-(9-isothiocyanatophenyl)pyridin-3-yl]oxy}methyl)cyclopropyl]-methylamine hydrochloride

The compound is obtained in accordance with the procedure of Step 2 of Example 1, using the compound obtained in the above Step 1.

Melting point (cap): 172-176° C.

Mass spectrometry (ESI) m/z=346.0770 Th ([M+H]⁺)

### EXAMPLE 88

### Methyl{1-[({5-[3-(2H-tetrazol-5-yl)phenyl]pyridin-3-yl}oxy)methyl]cyclopropyl}-amine dihydrochloride

### Step 1: tert-Butyl methyl{1-([({5-[3-(2H-tetrazol-5-yl)phenyl]pyridin-3-yl}oxy)methyl]-cyclopropyl}carbamate

1.2 g of azidotrimethyltin is added to a solution of 0.7 g of the compound obtained in Step 1 of Example 21 in 20 ml of toluene. The reaction mixture is heated at reflux for 20 hours and then concentrated. Chromatography on silica gel (dichloromethane/methanol: 95/5) allows 0.7 g of the expected product to be obtained.

### Step 2: Methyl{1-[({5-[3-(2H-tetrazol-5-yl)phenyl]pyridin-3-yl}oxy)methyl]cyclopropyl}amine dihydrochloride

10 ml of 4N hydrochloric acid solution in dioxane are added to a solution of 0.65 g of the compound obtained in the above Step 1 in 10 ml of ethanol. After stirring for 20 hours, the reaction mixture is diluted with ether and then filtered. The solid collected is taken up in water and the aqueous solution is lyophilised to obtain 0.49 g of the expected product.

Mass spectrometry (ESI) m/z=323.1595 Th ([M+H]⁺)

### EXAMPLE 89

### Methyl{1-[({5-[4-(2H-tetrazol-5-yl)phenyl]pyridin-3-yl}oxy)methyl]cyclopropyl}-amine dihydrochloride

### Step 1: tert-Butyl methyl{1-[({5-[4-(2H-tetrazol-5-yl)phenyl]pyridin-3-yl}oxy)methyl]-cyclopropylcarbamate

The compound is obtained in accordance with the procedure of Step 1 of Example 88, using the compound of Step 1 of Example 20 instead of the compound of Step 1 of Example 21.

### Step 2: Methyl{1-[({5-[4-(2H-tetrazol-5-yl)phenyl]pyridin-3-yl}oxy)methyl]cyclopropyl}amine dihydrochloride

The compound is obtained in accordance with the procedure of Step 2 of Example 88, using the compound obtained in the above Step 1.

Mass spectrometry (ESI) m/z=323.1610 Th ([M+H]⁺)

### EXAMPLE 90

### {1-[({6-Chloro-5-[4-(2H-tetrazol-5-yl)phenyl]pyridin-3-yl}oxy)methyl]cyclopropyl}-methylamine dihydrochloride

### Step 1: tert-Butyl{1-[({6-chloro-5-[4-(2H-tetrazol-5-yl)phenyl]pyridin-3-yl}oxy)-methyl]cyclopropyl}methylcarbamate

The compound is obtained in accordance with the procedure of Step 1 of Example 88, using the compound of Step 1 of Example 18 instead of the compound of Step 1 of Example 21.

### Step 2: {1-[({6-Chloro-5-[4-(2H-tetrazol-5-yl)phenyl]pyridin-3yl}oxy)methyl]cyclopropyl}methylamine dihydrochloride

6 ml of 4N hydrochloric acid solution in dioxane are added to a solution of 0.84 g of the compound obtained in the above Step 1 in 15 ml of ethanol. After stirring for 20 hours, the reaction mixture is diluted with ether and then filtered to obtain, after drying the solid, 0.75 g of the expected product.

Melting point (cap): decomposition >160° C.

Mass spectrometry (ESI) m/z=357.1216 Th ([M+H]⁺)

### EXAMPLE 91

### {1-[({6-Chloro-5-[3-(2H-tetrazol-5-yl)phenyl]pyridin-3-yl}oxy)methyl]cyclopropyl}-methylamine dihydrochloride

### Step 1: 3-(2-Chloro-5-{[1-(methylamino)cyclopropyl]methoxy}pyridin-3-yl)benzonitrile

The compound is obtained in accordance with the procedure of Step 1 of Example 1, using the compound of Preparation 2 instead of the compound of Preparation 1 and with the replacement of (3-methoxyphenyl)boronic acid with (3-cyanophenyl)boronic acid.

### Step 2: tert-Butyl{1-[({6-chloro-5-[3-(2H-tetrazol-5-yl)phenyl]pyridin-3-yl}oxy)-methyl]cyclopropyl}methylcarbamate

The compound is obtained in accordance with the procedure of Step 1 of Example 88, using the compound obtained in the above Step 1.

### Step 3: {1-[({6-Chloro-5-[3-(2H-tetrazol-5-yl)phenyl]pyridin-3-yl}oxy)methyl]cyclopropyl}methylamine dihydrochloride

The compound is obtained in accordance with the procedure of Step 2 of Example 88, using the compound obtained in the above Step 2.

Mass spectrometry (ESI) m/z=357.1230 Th ([M+H]⁺)

### EXAMPLE 92

### (1S,2R),(1R,2S)—N,2-Dimethyl-1-({[5-(2-methylphenyl)pyridin-3-yl]oxy}methyl)-cyclopropanamine dihydrochloride

0.17 g of tetrakis(triphenylphosphine)palladium are added under nitrogen to a solution of 1 g of the compound of Preparation 6 in 20 ml of toluene. The mixture is stirred for 20 minutes and then a solution of 0.61 g of (2-methylphenyl)boronic acid in 10 ml of ethanol and 10 ml of saturated aqueous sodium hydrogen carbonate solution are added. The reaction mixture is heated for 12 hours at 80° C. and then filtered and decanted. The organic phase is dried over sodium sulphate and concentrated. The residue is chromatographed on an RP18 column, 12-25μ (water/trifluoroacetic acid: 1000/2.5 to water/acetonitrile/trifluoroacetic acid: 800/200/2.5). The chromatography fractions are combined and then the acetonitrile is evaporated off. The residual aqueous solution is neutralised and then saturated with solid sodium hydrogen carbonate and subsequently extracted with dichloromethane. After drying over sodium sulphate and concentrating the organic phase, 0.57 g of the base obtained are dissolved in 10 ml of ethanol and 1.35 ml of 4N hydrochloric acid solution in dioxane is added. After concentration and crystallisation in ether, filtration and drying are carried out to obtain 0.51 g of the expected product.

Melting point (cap): 198-201° C.

Mass spectrometry (ESI) m/z=282 Th ([M+H]⁺)

### EXAMPLE 93

### (1S,2R),(1R,2S)-1-[({5-[3,5-bis(Trifluoromethyl)phenyl]pyridin-3-yl}oxy)methyl]-N,2-dimethylcyclopropanamine dihydrochloride

The compound is obtained in accordance with the procedure of Example 92, with the replacement of (2-methylphenyl)boronic acid with [3,5-bis(trifluoromethyl)phenyl]boronic acid.

Melting point (cap): 117-120° C.

Mass spectrometry (ESI) m/z=405.1418 Th ([M+H]⁺)

### EXAMPLE 94

### (1S,2R),(1R,2S)—N,2-Dimethyl-1-({[5-(2,3,4-trimethoxyphenyl)pyridin-3-yl]oxy}-methyl)cyclopropanamine dihydrochloride

The compound is obtained in accordance with the procedure of Example 92, with the replacement of (2-methylphenyl)boronic acid with (2,3,4-trimethoxyphenyl)boronic acid.

Melting point (cap): 196-199° C.

Mass spectrometry (ESI) m/z=359.1964 Th ([M+H]⁺)

### EXAMPLE 95

### [1-({[5,6-bis(4-Chlorophenyl)pyridin-3-yl]oxy}methyl)cyclopropyl]methylamine dihydrochloride

### Step 1: tert-Butyl[1-({[5,6-bis(4-chlorophenyl)pyridin-3-yl]oxy}methyl)cyclopropyl]-methylcarbamate

The compound, a product of disubstitution, is obtained during the procedure of Step 1 of Example 13.

### Step 2: [1-({[5,6-bis(4-Chlorophenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 220-225° C.

Mass spectrometry (ESI) m/z=399.1033 Th ([M+H]⁺)

### EXAMPLE 96

### [1-({[5,6-bis(4-Nitrophenyl)pyridin-3-yl]oxy}methyl)cyclopropyl]methylamine dihydrochloride

### Step 1: tert-Butyl[1-({[5,6-bis(4-nitrophenyl)pyridin-3-yl]oxy}methyl)cyclopropyl]-methylcarbamate

The compound, a product of disubstitution, is obtained during the procedure of Step 1 of Example 12.

### Step 2: [1-({[5,6-bis(4-Nitrophenyl)pyridin-3-yl]oxy}methyl)cyclopropyl]methylamine dihydrochloride

The compound is obtained in accordance with the procedure of Step 2 of Example 2, using the compound obtained in the above Step 1.

Melting point (cap): 215-220° C.

Mass spectrometry (ESI) m/z=421.1500 Th ([M+H]⁺)

### EXAMPLE 97

### 4,4′-(5-{[1-(Methylamino)cyclopropyl]methoxy}pyridine-2,3-diyl)dibenzonitrile dihydrochloride

### Step 1: tert-Butyl[1-({5,6-bis(4-cyanophenyl)pyridin-3-yl]oxy}methyl)cyclopropyl]-methylcarbamate

The compound, a product of disubstitution, is obtained during the procedure of Step 1 of Example 18.

### Step 2: 4,4′-(5-{[1-(Methylamino)cyclopropyl]methoxy}pyridine-2,3-diyl)dibenzonitrile dihydrochloride

The compound is obtained in accordance with the procedure of Step 2 of Example 2, using the compound obtained in the above Step 1.

Melting point (cap): 205-208° C.

Mass spectrometry (ESI) m/z=381.1718 Th ([M+H]⁺)

### EXAMPLE 98

### [1-({[5-(4-Aminophenyl)-6-methylpyridin-3-yl]oxy}methyl)cyclopropyl]methylamine trihydrochloride

### Step 1: tert-Butyl[1-({[5-(4-aminophenyl)-6-methylpyridin-3-yl]oxy}methyl)cyclopropyl]methylcarbamate

The compound is obtained in accordance with the procedure of Step 1 of Example 1, using the compound of Preparation 3 instead of the compound of Preparation 1 and with the replacement of (3-methoxyphenyl)boronic acid with (4-aminophenyl)boronic acid.

### Step 2: [1-({[5-(4-Aminophenyl)-6-methylpyridin-3-yl]oxy}methyl)cyclopropyl]methylamine trihydrochloride

40 ml of dioxane and then 10 ml of 4N hydrochloric acid solution in dioxane are added to 1.05 g of the product obtained in the above Step 1. 40 ml of ethanol are added to effect complete homogenisation of the reaction mixture. After stirring for 20 hours, the solvents are evaporated off and the residue is taken up in a minimum of ethanol. After dilution with ether, filtration and drying, 0.9 g of the expected product is obtained.

Melting point (cap): 245-250° C.

Mass spectrometry (ESI) m/z=284.169 Th ([M+H]⁺)

### EXAMPLE 99

### 3-[5-({[1-(Methylamino)cyclopropyl]methyl}amino)pyridin-3-yl]benzoic acid dihydrochloride

### Step 1: 3-{5-[({1-[(tert-Butoxycarbonyl)(methyl)amino]cyclopropyl}methyl)-amino]pyridin-3-yl}benzoic acid

A solution containing 3.96 g of sodium carbonate in 94 ml of water and then 2.05 g of 3-carboxyphenylboronic acid are added in succession to a mixture containing 140 ml of acetonitrile and 4.68 g of the compound of Preparation 8. The mixture is stirred for one hour under nitrogen and then 0.55 g of tetrakis(triphenylphosphine)palladium is added. Stirring is carried out for 1 hour at 20° C. and then for 20 hours at reflux. The mixture is concentrated to dryness, taken up in 40 ml of water and extracted repeatedly with ether. The aqueous phase is acidified with N hydrochloric acid until a pH of 5.5 is reached. Extraction with dichloromethane, drying over sodium sulphate and concentration to dryness are carried out. Chromatography on silica gel (dichloromethane/methanol: 95/5) allows 2.4 g of the expected product to be obtained.

Melting point (cap): 109° C.

### Step 2: 3-[5-({[1-(Methylamino)cyclopropyl]methyl}amino)pyridin-3-yl]benzoic acid dihydrochloride

2.26 g of the compound obtained in the above Step 1 are dissolved in 44 ml of dioxane. 44 ml of 4M hydrochloric acid in dioxane are added and then, after stirring for one hour, 7.3 ml of water are added. Stirring is carried out for 20 hours. Filtration with suction is carried out, and the precipitate is washed with ether and dried at 60° C. under 0.5 ton. 1.96 g of the expected product are obtained.

Melting point (cap): 248-250° C.

Mass spectrometry (ESI) (H₂O/CH₃CN) m/z=298.1527 Th ([M+H]⁺)

### EXAMPLE 100

### 4-[5-({[1-Methylamino)cyclopropyl]methyl}amino)pyridin-3-yl]benzoic acid dihydrochloride

### Step 1: 4-{5-[({1-[(tert-Butoxycarbonyl)(methyl)amino]cyclopropyl}methyl)amino]-pyridin-3-yl}benzoic acid

The compound is obtained in accordance with the procedure of Step 1 of Example 99, with the replacement of 3-carboxyphenylboronic acid with 4-carboxyphenylboronic acid.

Melting point (cap): 120° C.

### Step 2: 4-[5-({[1-Methylamino)cyclopropyl]methyl}amino)pyridin-3-yl]benzoic acid dihydrochloride

The compound is obtained in accordance with the procedure of Step 2 of Example 99, using the compound obtained in the above Step 1.

Melting point (cap): 256-260° C.

Mass spectrometry (ESI) (H₂O/CH₃CN) m/z=298.1522 Th ([M+H]⁺)

### EXAMPLE 101

### 4-[5-(Methyl{[1-(methylamino)cyclopropyl]methyl}amino)pyridin-3-yl]benzoic acid dihydrochloride

### Step 1: 4-{5-[{1-[(tert-Butoxycarbonyl)(methyl)amino]cyclopropyl}methyl)-(methyl)amino]pyridin-3-yl}benzoic acid

The compound is obtained in accordance with the procedure of Step 1 of Example 99, using the compound of Preparation 9 instead of the compound of Preparation 8 and with the replacement of 3-carboxyphenylboronic acid with 4-carboxyphenylboronic acid.

### Step 2: 4-[5-(Methyl{[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Mass spectrometry (ESI) (H₂O/CH₃CN) m/z=312.1676 Th ([M+H]⁺)

### EXAMPLE 102

### 3-[5-(Methyl{[1-(methylamino)cyclopropyl]methyl}amino)pyridin-3-yl]benzoic acid dihydrochloride

### Step 1: 3-[5-[({1-[tert-Butoxycarbonyl)(methyl)amino]cyclopropyl}methyl)(methyl)-amino]pyridin-3-yl]benzoic acid

The compound is obtained in accordance with the procedure of Step 1 of Example 99, using the compound of Preparation 9 instead of the compound of Preparation 8.

### Step 2: 3-[5-(Methyl{[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Mass spectrometry (ESI) (H₂O/CH₃CN) m/z=312.1689 Th ([M+H]⁺)

### EXAMPLE 103

### 2-Chloro-N-{[1-(methylamino)cyclopropyl]methyl}-3,3′-bipyridin-5-amine trihydrochloride

### Step 1: tert-Butyl 1-{[(2-chloro-3,3′-bipyridin-5-yl)amino]methyl}cyclopropyl)-methylcarbamate

A mixture composed of 1 g of the compound of Preparation 10, 25 ml of toluene and 0.15 g of tetrakis(triphenylphosphine)palladium is stirred for one hour at 20° C. There are added in succession 0.39 g of 3-pyridineboronic acid, 12.5 ml of ethanol and 12.5 ml of saturated aqueous sodium hydrogen carbonate solution. The mixture is heated for 20 hours at 80° C. with active stirring. After cooling, toluene is added, decanting is carried out, and the organic phase is dried over sodium sulphate and concentrated to dryness. Chromatography on silica gel (dichloromethane/tetrahydrofuran: 97/3) allows 0.99 g of the expected product to be obtained.

### Step 2: 2-Chloro-N-{[1-(methylamino)cyclopropyl]methyl}-3,3′-bipyridin-5-amine trihydrochloride

1.0 g of the product obtained in the above Step 1 is stirred for 20 hours at ambient temperature with 50 ml of ethanol and 10 ml of 4N hydrochloric acid in dioxane. Dilution with ether is carried out, followed by filtration with suction and drying at 60° C. under 1 torr. 0.78 g of the desired product is obtained.

Melting point (cap): 180-185° C.

Mass spectrometry (ESI) (H₂O/CH₃CN) m/z=289.1219 Th ([M+H]⁺)

### EXAMPLE 104

### 3-[2-Chloro-5-({[1-(methylamino)cyclopropyl]methyl}amino)pyridin-3-yl]benzoic acid dihydrochloride

### Step 1: 3-{5-[({1-[(tert-Butoxycarbonyl)(methyl)amino]cyclopropyl}methyl)amino]-2-chloropyridin-3-yl}benzoic acid

The compound is obtained in accordance with the procedure of Step 1 of Example 99, using the compound of Preparation 10 instead of the compound of Preparation 8.

### Step 2: 3-[2-Chloro-5-({[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Melting point (cap): 140-145° C.

Mass spectrometry (ESI) (H₂O/CH₃CN) m/z=332.1 Th ([M+H]+)

### EXAMPLE 105

### 4-[2-Chloro-5-({[1-(methylamino)cyclopropyl]methyl}amino)pyridin-3-yl]benzoic acid dihydrochloride

### Step 1: 4-{5-[({1-[(tert-Butoxycarbonyl)methyl)amino]cyclopropyl}methyl)amino]-2-chloropyridin-3-yl}benzoic acid

The compound is obtained in accordance with the procedure of Step 1 of Example 99, using the compound of Preparation 10 instead of the compound of Preparation 8 and with the replacement of 3-carboxyphenylboronic acid with 4-carboxyphenylboronic acid.

### Step 2: 4-[2-Chloro-5-({[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Melting point (cap): 175-182° C.

Mass spectrometry (ESI) (H₂O/CH₃CN) m/z=332.1147 Th ([M+H]+)

### EXAMPLE 106

### 2-Chloro-N-{[1-(methylamino)cyclopropyl]methyl}-3,4′-bipyridin-5-amine dihydrochloride

### Step 1: tert-Butyl (1-{[(2-chloro-3,4′bipyridin-5-yl)amino]methyl}cyclopropyl)-methylcarbamate

The compound is obtained in accordance with the procedure of Step 1 of Example 103, with the replacement of 3-pyridineboronic acid with 4-pyridineboronic acid.

### Step 2: 2-Chloro-N-{[1-(methylamino)cyclopropyl]methyl}-3,4′-bipyridin-5-amine dihydrochloride

The compound is obtained in accordance with the procedure of Step 2 of Example 103, using the compound obtained in the above Step 1.

Melting point (cap): 100-110° C.

Mass spectrometry (ESI) (H₂O/CH₃CN) m/z=289.1219 Th ([M+H]⁺)

### EXAMPLE 107

### 6-Chloro-5-(4-chlorophenyl)-N-{[1-(methylamino)cyclopropyl]methyl}pyridin-3-amine dihydrochloride

### Step 1: tert-Butyl[1-({[6-chloro-5-(4-chlorophenyl)pyridin-3-yl]amino}methyl)cyclo-propyl]methylcarbamate

The compound is obtained in accordance with the procedure of Step 1 of Example 103, with the replacement of 3-pyridineboronic acid with (4-chlorophenyl)boronic acid.

### Step 2: 6-Chloro-5-(4-chlorophenyl)-N-{[1-(methylamino)cyclopropyl]methyl}pyridin-3-amine dihydrochloride

18 ml of 4N hydrochloric acid in dioxane are added to 2 g of the product obtained in the above Step 1 dissolved in 80 ml of dioxane. The whole is stirred for 16 hours at 20° C. and diluted with 80 ml of ether. Stirring is carried out for 1 hour, and the precipitate is filtered off with suction and dried at 50° C. under 1 ton. 1.57 g of the expected product are obtained.

Melting point (cap): 128-139° C.

Mass spectrometry (ESI) (H₂O/CH₃CN) m/z=332.0876 Th ([M+H]⁺)

### EXAMPLE 108

### 5-(4-Aminophenyl)-6-chloro-N-{[1-(methylamino)cyclopropyl]methyl}pyridin-3-amine dihydrochloride

### Step 1: tert-Butyl[1-({[5-(4-aminophenyl)-6-chloropyridin-3-yl]amino}methyl)cyclopropyl]methylcarbamate

Under a nitrogen atmosphere, a mixture consisting of 2.0 g of the compound of Preparation 10, 0.25 g of tetrakis(triphenylphosphine)palladium and 1.33 g of 444,4,5,5-tetramethyl-1,3,2-dioxaborolan-2-yl)aniline in 60 ml of THF is stirred for 30 minutes. 3.2 g of potassium carbonate dissolved in 20 ml of water are added. Stirring is carried out for 20 hours at 60° C. Concentration to dryness is carried out. The residue is taken up in aqueous sodium carbonate solution and extracted with dichloromethane. The dichloromethane phase is dried over sodium sulphate and concentrated to dryness. Chromatography on silica gel (dichloromethane/butanone: 93/7) allows 1.67 g of the expected product to be obtained.

### Step 2: 5-(4-Aminophenyl)-6-chloro-N-{[1-(methylamino)cyclopropyl]methyl}pyridin-3-amine dihydrochloride

The compound is obtained in accordance with the procedure of Step 2 of Example 103, using the compound obtained in the above Step 1.

Melting point (cap): 190-196° C.

Mass spectrometry (ESI) (H₂O/CH₁CN) m/z=303.1 Th ([M+H]⁺)

### EXAMPLE 109

### 4-[2-Chloro-5-({[1-(methylamino)cyclopropyl]methyl}amino)pyridin-3-yl]benzonitrile hydrochloride

### Step 1: tert-Butyl[1-({[6-chloro-5-(4-cyanophenyl)pyridin-3-yl]amino}methyl)cyclopropyl]methylcarbamate

The compound is obtained in accordance with the procedure of Step 1 of Example 103, with the replacement of 3-pyridineboronic acid with (4-cyanophenyl)boronic acid.

### Step 2: 4-[2-Chloro-5-({[1-(methylamino)cyclopropyl]methyl}amino)pyridin-3-yl]benzonitrile hydrochloride

The compound is obtained in accordance with the procedure of Step 2 of Example 107, using the compound obtained in the above Step 1.

Mass spectrometry (ESI) (H₂O/CH₃CN) m/z=313.1 Th ([M+H]⁺)

### EXAMPLE 110

### 4-[2-Chloro-5-(methyl{[1-(methylamino)cyclopropyl]methyl}amino)pyridin-3-yl]benzoic acid dihydrochloride

### Step 1: 4-{5-[({1-[(tert-Butoxycarbonyl)(methyl)amino]cyclopropyl}methyl(methyl)-amino]-2-chloropyridin-3-yl}benzoic acid

The compound is obtained in accordance with the procedure of Step 1 of Example 99, using the compound of Preparation 11 instead of the compound of Preparation 8 and with the replacement of 3-carboxyphenylboronic acid with 4-carboxyphenylboronic acid.

### Step 2: 4-[2-Chloro-5-(methyl{[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Melting point (cap): 138-145° C.

Mass spectrometry (ESI) (H₂O/CH₃CN) m/z=346.1 Th ([M+H]⁺)

### EXAMPLE 111

### 2-Chloro-N-methyl-N-{[1-(methylamino)cyclopropyl]methyl}-3,4′-bipyridin-5-amine dihydrochloride

### Step 1: tert-Butyl(1-{[(2-chloro-3,4′-bipyridin-5-yl)(methyl)amino]methyl}-cyclopropyl)methylcarbamate

The compound is obtained in accordance with the procedure of Step 1 of Example 103, using the compound of Preparation 11 instead of the compound of Preparation 10 and with the replacement of 3-pyridineboronic acid with 4-pyridineboronic acid.

### Step 2: 2-Chloro-N-methyl-N-{[1-(methylamino)cyclopropyl]methyl}-3,4′-bipyridin-5-amine dihydrochloride

The compound is obtained in accordance with the procedure of Step 2 of Example 103, using the compound obtained in the above Step 1.

Melting point (cap): 110-120° C.

Mass spectrometry (ESI) (H₂O/CH₃CN) m/z=303.1 Th ([M+H]⁺)

### EXAMPLE 112

### 4-[2-Methyl-5-({[1-(methylamino)cyclopropyl]methyl}amino)pyridin-3-yl]benzoic acid dihydrochloride

### Step 1: 4-{5-[({1-[(tert-Butoxycarbonyl)(methyl)amino]cyclopropyl}methyl)amino]-2-methylpyridin-3-yl}benzoic acid

The compound is obtained in accordance with the procedure of Step 1 of Example 99, using the compound of Preparation 12 instead of the compound of Preparation 8 and with the replacement of 3-carboxyphenylboronic acid with 4-carboxyphenylboronic acid. Melting point (cap): 146° C.

### Step 2: 4-[2-Methyl-5-({[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Melting point (cap): 241-245° C.

Mass spectrometry (ESI) (H₂O/CH₃CN) m/z=312.2 Th ([M+H]⁺)

### EXAMPLE 113

### 2-Methyl-N-{[1-(methylamino)cyclopropyl]methyl}-3,4′-bipyridin-5-amine trihydrochloride

### Step 1: tert-Butyl methyl(1-{[(2-methyl-3,4′-bipyridin-5-yl)amino]methyl}cyclopropyl)-carbamate

The compound is obtained in accordance with the procedure of Step 1 of Example 103, using the compound of Preparation 12 instead of the compound of Preparation 10 and with the replacement of 3-pyridineboronic acid with 4-pyridineboronic acid.

### Step 2: 2-Methyl-N-{[1-(methylamino)cyclopropyl]methyl}-3,4′-bipyridin-5-amine trihydrochloride

The compound is obtained in accordance with the procedure of Step 2 of Example 103, using the compound obtained in the above Step 1.

Melting point (cap): 240-248° C.

Mass spectrometry (ESI) (H₂O/CH₃CN) m/z=269.2 Th ([M+H]⁺)

### EXAMPLE 114

### 5-(4-Aminophenyl)-6-methyl-N-{[1-(methylamino)cyclopropyl]methyl}pyridin-3-amine trihydrochloride

### Step 1: tert-Butyl[1-({[5-(4-aminophenyl)-6-methylpyridin-3-yl]amino}methyl)-cyclopropyl]methylcarbamate

The compound is obtained in accordance with the procedure of Step 1 of Example 108, using the compound of Preparation 12 instead of the compound of Preparation 10.

### Step 2: 5-(4-Aminophenyl)-6-methyl-N-({[1-(methylamino)cyclopropyl]methyl}pyridin-3-amine trihydrochloride

The compound is obtained in accordance with the procedure of Step 2 of Example 103, using the compound obtained in the above Step 1.

Melting point (cap): 190-200° C.

Mass spectrometry (ESI) (H₂O/CH₃CN) m/z=283.2 Th ([M+H]⁺)

### EXAMPLE 115

### 4-[2-Methyl-5-(methyl{[1-(methylamino)cyclopropyl]methyl}amino)pyridin-3-yl]benzoic acid dihydrochloride

### Step 1: 4-{5-[({1-[(tert-Butoxycarbonyl)(methyl)amino]cyclopropyl}methyl(methyl)-amino]-2-methylpyridin-3-yl}benzoic acid

The compound is obtained in accordance with the procedure of Step 1 of Example 99, using the compound of Preparation 13 instead of the compound of Preparation 9 and with the replacement of 3-carboxyphenylboronic acid with 4-carboxyphenylboronic acid.

### Step 2: 4-[2-Methyl-5-(methyl{[1-(methylamino)cyclopropyl]methyl}amino)pyridin-3-yl]benzoic acid dihydrochloride

The compound is obtained in accordance with the procedure of Step 2 of Example 99, using the compound obtained in the above Step 1.

Mass spectrometry (ESI) (H₂O/CH₃CN) m/z=326.1870 Th ([M+H]⁺)

### EXAMPLE 116

### N,2-Dimethyl-N-{[1-(methylamino)cyclopropyl]methyl}-3,4′-bipyridin-5-amine trihydrochloride

### Step 1: tert-Butyl methyl(1{[methyl(2-methyl-3,4′-bipyridin-5-yl)amino]methyl}cyclopropyl)carbamate

The compound is obtained in accordance with the procedure of Step 1 of Example 103, using the compound of Preparation 13 instead of the compound of Preparation 10 and with the replacement of 3-pyridineboronic acid with 4-pyridineboronic acid.

### Step 2: N,2-Dimethyl-N-{[1-(methylamino)cyclopropyl]methyl}-3,4′-bipyridin-5-amine trihydrochloride

The compound is obtained in accordance with the procedure of Step 2 of Example 103, using the compound obtained in the above Step 1.

Melting point (cap): 182-187° C.

Mass spectrometry (ESI) (H₂O/CH₁CN) m/z=283.2 Th ([M+H]⁺)

### EXAMPLE 117

### 5-(4-Aminophenyl)-N,6-dimethyl-N-{[1-(methylamino)cyclopropyl]methyl}pyridin-3-amine trihydrochloride

### Step 1: tert-Butyl(1-{[[5-(4-aminophenyl)-6-methylpyridin-3-yl](methyl)amino]-methyl}cyclopropyl)methylcarbamate

The compound is obtained in accordance with the procedure of Step 1 of Example 108, using the compound of Preparation 13 instead of the compound of Preparation 10.

### Step 2: 5-(4-Aminophenyl)-N,6-dimethyl-N-{[1-(methylamino)cyclopropyl]-methyl}pyridin-3-amine trihydrochloride

The compound is obtained in accordance with the procedure of Step 2 of Example 103, using the compound obtained in the above Step 1.

Melting point (cap): 235-240° C.

Mass spectrometry (ESI) (H₂O/CH₃CN) m/z=297.2 Th ([M+H]⁺)

### Pharmacological Studies of Compounds of the Invention

### EXAMPLE A

### Displacement of Binding of [¹²⁵]-α-Bungarotoxin to Nicotinic Receptors of the Electric Organ of Torpedo Fish

This study, carried out according to the method described in J. Pharmacol. Exp. Ther., 1994, 271 ; 624-631, is aimed at assessing the affinity of compounds of the present invention for nicotinic receptors of the “muscular” type.

Membranes (1-5 μg/ml) of the electric organ of torpedo fish are incubated (1 hour, 22° C.) in the presence of a series of concentrations (0.01-10 μM) of each compound of the invention (diluted starting from a 10 mM stock solution in DMSO) in the presence of [¹²⁵]-α-bungarotoxin (S.A.: 7.4 TBq/mmol: 0.2 nM) in Krebs buffer (Tris-HCl 50 mM, KCl 5 mM, MgCl₂ 1 mM, CaCl₂ 2 mM, NaCl 100 mM, pH 7.4) with 0.01% BSA; final volume: 500 μl. The non-specific binding is determined by incubating membranes in the presence of a-bungarotoxin (1 μM).

The results show that, up to a concentration of 10 μM, the compounds of the present invention have no significant affinity for nicotinic receptors of the “muscular” type.

### EXAMPLE B

### Displacement of Binding of [³H]-Epibatidine to Nicotinic Receptors of IMR32 Cells

This study, carried out according to the technique described in Molec. Pharmacol., 1995, 48; 280-287, is aimed at determining the affinity of compounds of the present invention for nicotinic receptors of the “ganglionic” type (American Soc. Neuroscience, 2000, 26, 138).

Membranes (250 μg/ml) of IMR-32 neuroblastoma cells are incubated (2 hours, 20° C.) in the presence of a series of concentrations (0.01-10 μM) of each compound of the invention (diluted starting from a 10 mM stock solution in DMSO) and (±)[³H]epibatidine (S.A.: 2464 GBq/mmol: 1.5 nM) in phosphate buffer (NaH₂PO₄ 20 mM, pH 7.4); final volume: 250 μl. The non-specific binding is determined by incubating membranes in the presence of 300 μM of (−)nicotine.

The results show that, up to a concentration of 10 μM, the compounds of the present invention have no significant affinity for nicotinic receptors of the “ganglionic” type.

### EXAMPLE C

### Displacement of Binding of [³H]-Oxotremorine-M to Muscarinic Receptors of Rat Brain

This study, carried out according to the method described in Naumyn-Schmiederberg's Arch. Pharmacol., 2001, 363, 429-438, is aimed at determining the affinity of compounds of the present invention for muscarinic receptors.

Membranes (250 μg/ml) of rat brain are incubated (2 hours, 20° C.) in the presence of a series of concentrations (0.01-10 μM) of each compound of the invention (diluted starting from a 10 mM stock solution in DMSO) and [³H]-oxotremorine-M (S.A.: 3174 GBq/mmol: 2 nM) in phosphate buffer (NaH₂PO₄ 20 mM, pH 7.4); final volume: 250 μl. The specific binding is determined by incubating membranes in the presence of atropine (1 μM). The affinity of the compounds of the present invention for muscarinic receptors is characterised by determination of the Kᵢ.

The results show that, up to a concentration of 10 μM, the majority of the compounds of the present invention have no affinity for muscarinic receptors.

### EXAMPLE D

### Displacement of Binding of [¹²⁵]-α-bungarotoxin to “Type α7” Nicotinic Receptors of Rat Brain

This study, carried out according to the method described in Molec. Pharmacol., 1986, 30; 427-436, is aimed at determining the affinity of compounds of the present invention for type a7 central nicotinic receptors.

Membranes (1000 μg/ml) of rat brain are incubated (5 hours, 37° C.) in the presence of a series of concentrations (0.01-10 μM) of each compound of the present invention (diluted starting from a 10 mM stock solution in DMSO) and [¹²⁵]-α-bungarotoxin (S.A.: 7.4 TBq/mmol: 1 nM) in Krebs buffer (Tris-HCl 50 mM, KCl 5 mM, MgCl₂ 1 mM, CaCl₂ 2 mM, NaCl 100 mM, pH 7.4) with 0.05% BSA; final volume: 500 μl. The non-specific binding is determined by incubating membranes in the presence of α-bungarotoxin (1 μM). The affinity of compounds of the present invention for type a7 nicotinic receptors is characterised by determination of the Kᵢ.

The results indicate that, up to a concentration of 10 μM, the majority of the compounds of the present invention have no affinity for type α7 central nicotinic receptors. Some compounds of the invention have a Kᵢ of the order of 10 μM.

### EXAMPLE E

### Displacement of Binding of [³H]-cytisine to “type α4β2” Nicotinic Receptors of Rat Brain

This study, carried out according to the technique described in Molec. Pharmacol., 1990, 39; 9-12, is aimed at determining the affinity of compounds of the present invention for type α4β32 central nicotinic receptors.

Membranes (250 μg/ml) of rat brain are incubated (2 hours, 20° C.) in the presence of a series of concentrations (0.01-10 μM) of each compound of the present invention (diluted starting from a 10 mM stock solution in DMSO) and [³H]-cytisine (S.A.: 1184 GBq/mmol: 2 nM) in phosphate buffer (NaH₂PO₄ 20 mM, pH 7.4); final volume: 250 μl. The non-specific binding is determined by incubating membranes in the presence of 10 μM of (−)nicotine. The affinity of the compounds of the present invention for type α4β2 central nicotinic receptors is characterised by determination of the Kᵢ.

The results obtained show that the compounds of the present invention have a strong affinity for type α4β2 central nicotinic receptors, having Kᵢ values of the order of 1 nM.

These results, and also those obtained in Examples A to D, indicate that the compounds of the present invention are powerful central nicotinic ligands that are specific to type α4β2 receptors.

TABLE 1
Affinity (Ki, nM) of the compounds of the present
invention for type α₄β₂ receptors

| Examples | Ki (nM) |
|---|---|
| 1 | 7.9 |
| 2 | 8.5 |
| 6 | 2.3 |
| 10 | 0.7 |
| 11 | 0.8 |
| 26 | 0.3 |
| 32 | 0.4 |
| 52 | 1.4 |

### EXAMPLE F

### In vivo Measurement of the Release of Acetylcholine by Means of Intra-Cortical Microdialysis in the Conscious Wistar Rat

The systemic administration of nicotine and nicotinic agonists causes an increase, in vivo, of acetylcholine in various regions of the brain (Neurochem. Res., 1996, 21, 1181-1186; Eur. J. Pharmacol., 1998, 351, 181-188; Br. J. Pharmacol., 1999, 127, 1486-1494). A microdialysis probe is implanted in the median prefrontal cortex of male Wistar rats. Six or seven days after they have been implanted, the probes are perfused with Ringer's solution (NaCl 147 mM, KCl 2.7 mM, CaCl₂ 1.2 mM, MgCl₂ 1 mM, neostigmine 20 nM) at a flow rate of 1 μl/min, the animal being free to move. After 2 hours in the animal quarters, the product under test is administered by the intraperitoneal route. A group of control animals receives the solvent used for the product. The dialysates (30 μl) are then collected every 30 minutes for 4 hours in order to measure the extra-synaptic cortical concentrations of acetylcholine by means of HPLC with amperometric detection. The results are expressed in pg of acetylcholine/dialysate, and inter-group comparisons are carried out by means of variance analysis, using 2 factors (treatment×time), with measurements being repeated over time.

The results obtained show that, in vivo, the compounds of the present invention increase the cortical release of acetylcholine in a dose-dependent manner for active doses ranging from 1 to 10 mg/kg IP, indicating the α4β2-agonist character of the compounds of the present invention. For example, at a dose of 10 mg/kg IP, the compound of Example 1 increases the release of acetylcholine (+72%), and the compound of Example 18 increases the release by +104% at the same dose.

### EXAMPLE G

### Abdominal Contractions Induced by Phenyl-P-Benzoquinone (PBQ) in the NMRI Mouse

Intraperitoneal administration of an alcoholic solution of PBQ causes abdominal cramps in the mouse (Proc. Soc. Exp. Biol., 1957, 95, 729-731). The cramps are characterised by repeated contractions of the abdominal musculature, accompanied by extension of the hind limbs. Most analgesics antagonise these abdominal cramps (Brit. J. Pharmacol. Chem., 1968, 32, 295-310). At t=0 min., the animals are weighed and the compound being studied is administered by the IP route. A group of control animals is given the solvent used for the compound. At t=30 min., an alcoholic solution of PBQ (0.2%) is administered by the IP route in a volume of 0.25 ml/mouse. Immediately after administration of the PBQ, the animals are placed in cylinders of plexiglass (L=19.5 cm; I.D.=5 cm). From t=35 min. to t=45 min., the animals' reaction is observed and the experimenter notes the total number of abdominal cramps per animal. The results are expressed as the percentage inhibition of the number of abdominal cramps measured in the control animals at the active dose of the compound studied.

The results obtained show inhibition ranging from −80% for active doses ranging from 10 mg/kg IP. This demonstrates that the compounds of the invention possess antalgic properties. For example, at a dose of 10 mg/kg IP, compounds 81 and 106 inhibit abdominal cramps by −90% and −87%, respectively.

### EXAMPLE H

### Social Recognition in the Wistar Rat

Initially described in 1982 (J. Comp. Physiol., 1982, 96, 1000-1006), the social recognition test has subsequently been proposed by various authors (Psychopharmacology, 1987, 91, 363-368; Psychopharmacology, 1989, 97, 262-268) for studying the mnemocognitive effects of new compounds. The test is based on the natural expression of the olfactory memory of the rat and its natural tendency to forget and allows evaluation of memorisation of an adult rat by recognition of a young congeneric animal. A young rat (21 days), taken at random, is placed for 5 minutes in the cage housing an adult rat. With the aid of a video device, the experimenter observes the social recognition behaviour of the adult rat and measures its overall duration. The young rat is then removed from the adult rat's cage and is placed in its own cage until the second introduction. The adult rat is given the compound under test by the intraperitoneal route and, after 2 hours, is again brought into the presence (5 minutes) of the young rat. The social recognition behaviour is then observed again and its duration measured. The assessment criterion is the difference (T2−T1), expressed in seconds, between the “recognition” times of the 2 encounters.

The results obtained show a difference (T2−T1) ranging from −16 s to −26 s for doses ranging from 3 to 10 mg/kg IP. This demonstrates that the compounds of the invention very greatly enhance memorisation, even at a low dose. The results obtained show a difference (T2−T1) of between −16 and −26 for doses ranging from 3 to 10 mg/kg IP for the compound of Example 1.

### EXAMPLE I

### Pharmaceutical Compositions for 1000 Tablets Each Containing 100 mg of Active Ingredient

| | |
|---|---|
| Compound of Example 1 | 10 g |
| Hydroxypropyl methyl cellulose | 10 g |
| Wheat starch | 15 g |
| Lactose | 90 g |
| Magnesium stearate | 2 g |

1-15. (canceled)

16. A compound selected from those of formula (I): 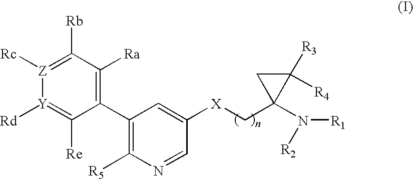
wherein:
n represents an integer of from 1 to 6 inclusive,
X represents an oxygen atom or an NR₆ group,
Y represents a carbon atom or a nitrogen atom, wherein when Y represents a nitrogen atom Rd is absent,
Z represents a carbon atom or a nitrogen atom, wherein when Z represents a nitrogen atom Rc is absent,
R₁ and R₂, which may be identical or different, each independently of the other represent a hydrogen atom, a linear or branched (C₁-C₆)alkyl group or an aryl-(C₁-C₆)alkyl group in which the alkyl moiety may be linear or branched,
R₃ and R₄, which may be identical or different, each independently of the other represent a hydrogen atom or a linear or branched (C₁-C₆)alkyl group,
R₅ represents a hydrogen atom, a linear or branched (C₁-C₆)alkyl, halogen, hydroxy, linear or branched (C₁-C₆)alkoxy, cyano, nitro, linear or branched (C₂-C₆)acyl, linear or branched (C₁-C₆)alkoxycarbonyl, linear or branched (C₁-C₆)trihaloalkyl or linear or branched (C₁-C₆)trihaloalkoxy, an amino group, optionally substituted by one or two linear or branched (C₁-C₆)alkyl groups, an aryl group or a heteroaryl group,
R₆ represents a hydrogen atom, a linear or branched (C₁-C₆)alkyl group or an aryl-(C₁-C₆)alkyl group in which the alkyl moiety may be linear or branched,
Ra, Rb, Rc, Rd and Re, which may be identical or different, each independently of the others represent a hydrogen atom, a linear or branched (C₁-C₆)alkyl, halogen, linear or branched (C₁-C₆)haloalkyl, hydroxy, linear or branched (C₁-C₆)alkoxy, linear or branched (C₁-C₆)hydroxyalkyl, cyano, nitro, carboxy, isothiocyanate, linear or branched (C₂-C₆)acyl, linear or branched (C₁-C₆)alkoxycarbonyl, linear or branched (C₁-C₆)trihaloalkyl, linear or branched (C₁-C₆)trihaloalkoxy, linear or branched (C₁-C₆)alkylthio, a (C₁-C₆)alkylcarbonylamino group in which the alkyl moiety may be linear or branched, a halo-(C₁-C₆)alkylcarbonylamino group in which the alkyl moiety may be linear or branched, an aminocarbonyl group, an amino group, optionally substituted by one or two linear or branched (C₁-C₆)alkyl groups, or a tetrazolyl group,
it being understood that:
aryl group means a phenyl, biphenyl, naphthyl, dihydronaphthyl, tetrahydronaphthyl, indanyl or indenyl group, each of those groups being optionally substituted by one or more identical or different groups selected from halogen atoms, linear or branched (C₁-C₆)alkyl, hydroxy, cyano, nitro, linear or branched (C₁-C₆)alkoxy, linear or branched (C₂-C₇)acyl, linear or branched (C₁-C₆)alkoxycarbonyl, linear or branched (C₁-C₆)trihaloalkyl, linear or branched (C₁-C₆)trihaloalkoxy, and amino groups, optionally substituted by one or two linear or branched (C₁-C₆)alkyl groups, and
heteroaryl group means an aromatic monocyclic system or a bicyclic system having from 5 to 12 chain members and containing from one to three identical or different hetero atoms selected from oxygen, nitrogen and sulphur, wherein one of the rings, in the case of the bicyclic system, has an aromatic character while the other ring may be aromatic or partially hydrogenated, and wherein each of those groups may optionally be substituted by one or more identical or different groups selected from halogen atoms, linear or branched (C₁-C₆)alkyl, hydroxy, cyano, nitro, linear or branched (C₁-C₆)alkoxy, linear or branched (C₂-C₇)acyl, linear or branched (C₁-C₆)alkoxycarbonyl, linear or branched (C₁-C₆)trihaloalkyl, linear or branched (C₁-C₆)trihaloalkoxy, and amino groups, optionally substituted by one or two linear or branched (C₁-C₆)alkyl groups,
its enantiomers, diastereoisomers, and addition salts thereof with a pharmaceutically acceptable acid or base.

17. The compound of claim 16, which is selected from those of formula (I/A):

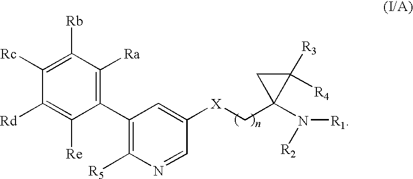

18. The compound of claim 16, which is selected from those of formula (I/B):

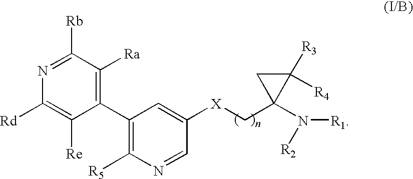

19. The compound of claim 16, which is selected from those of formula (I/C):

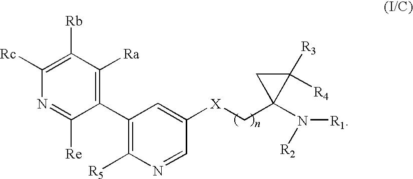

20. The compound of claim 16, wherein n is an integer having the value 1.

21. The compound of claim 16, wherein R₁ and R₂, which may be identical or different, each independently of the other represent a hydrogen atom or a linear or branched (C₁-C₆)alkyl group.

22. The compound of claim 16, wherein R₃ and R₄, which may be identical or different, each represent a hydrogen atom or a methyl group.

23. The compound of claim 16, wherein R₅ represents a hydrogen atom, a halogen atom or a methyl group.

24. The compound of claim 16, wherein R₆ represents a hydrogen atom or a methyl group.

25. The compound of claim 16 which is selected from:
[1-({[5-(3-methoxyphenyl)pyridin-3-yl]oxy}methyl)cyclopropyl]methylamine dihydrochloride,
[1-({[6-chloro-5-(3-methoxyphenyl)pyridin-3-yl]oxy}methyl)cyclopropyl]methylamine dihydrochloride,
[1-({[5-(4-methoxyphenyl)pyridin-3-yl]oxy}methyl)cyclopropyl]methylamine dihydrochloride,
[1-({[5-(4-chlorophenyl)pyridin-3-yl]oxy}methyl)cyclopropyl]methylamine hydrochloride,
[1-({[6-chloro-5-(4-fluorophenyl)pyridin-3-yl]oxy}methyl)cyclopropyl]methylamine hydrochloride,
{1-[({6-chloro-5-[4-(methylthio)phenyl]pyridin-3-yl}oxy)methyl]cyclopropyl}-methylamine dihydrochloride,
[1-({[6-chloro-5-(3,5-dichlorophenyl)pyridin-3-yl]oxy}methyl)cyclopropyl]methylamine hydrochloride,
N-[3-(2-chloro-5-{[1-(methylamino)cyclopropyl]methoxy}pyridin-3-yl)phenyl]acetamide hydrochloride,
ethyl 4-(2-chloro-5-{[1-(methylamino)cyclopropyl]methoxy}pyridin-3-yl)benzoate dihydrochloride,
4-(2-chloro-5-{[1-(methylamino)cyclopropyl]methoxy}pyridin-3-yl)benzamide hydrochloride,
4-(2-chloro-5-{[1-(methylamino)cyclopropyl]methoxy}pyridin-3-yl)benzoic acid hydrochloride,
(1-{[(2-chloro-3,4′-bipyridin-5-yl)oxy]methyl}cyclopropyl)methylamine dihydrochloride
{1-[({6-chloro-5-[4-(2H-tetrazol-5-yl)phenyl]pyridin-3-yl}oxy)methyl]cyclopropyl}-methylamine dihydrochloride,
[1-({[5,6-bis(4-chlorophenyl)pyridin-3-yl]oxy}methyl)cyclopropyl]methylamine dihydrochloride,
5-(4-aminophenyl)-6-methyl-N-{[1-(methylamino)cyclopropyl]methyl}pyridin-3-amine trihydrochloride,
and enantiomers, diastereoisomers, and addition salts thereof with a pharmaceutically acceptable acid or base.

26. A pharmaceutical composition comprising as active ingredient at least one compound according to claim 16, alone or in combination with one or more pharmaceutically acceptable, inert, non-toxic excipients or carriers.

27. A method of treating a living animal body, including a human, afflicted with a condition treatable by a specific nicotinic ligand of α4β2 receptors, comprising the step of administering to the living animal body, including a human, an amount of a compound of claim 16 which is effective for treatment of the condition.

28. The method of claim 27, wherein the condition is selected from deficiencies of memory associated with cerebral ageing and with neurodegenerative diseases, mood disorders, Tourette's syndrome, attention-deficit hyperactivity syndrome, tobacco withdrawal and pain.

29. The method of claim 27, wherein the condition is selected from deficiencies of memory associated with Alzheimer's disease, Parkinson's disease, Pick's disease, Korsakoff s disease and/or frontal lobe and subcortical dementias.
